(19) United States
(12) Patent Application Publication
(10) Pub. No.: US 2025/0361358 A1
(43) Pub. Date: Nov. 27, 2025

(54) ZINGEROL-BASED BIODEGRADABLE POLYESTERS AND 3D-PRINTABLE PHOTOPOLYMERIZABLE COMPOSITIONS, METHODS OF PREPARATION, AND USES THEREOF

(71) Applicant: Technion Research & Development Foundation Limited, Haifa (IL)

(72) Inventors: Shady FARAH, Haifa (IL); Simran JINDAL, Haifa (IL)

(21) Appl. No.: 19/215,991

(22) Filed: May 22, 2025

Related U.S. Application Data
(60) Provisional application No. 63/650,546, filed on May 22, 2024.

(51) Int. Cl. C08G 63/60; A01N 25/10; A01N 31/16; A01P 1/00; A61F 2/30; A61L 27/18; A61L 27/54; B33Y 10/00; B33Y 70/00; B33Y 80/00; C08F 22/20; C08F 22/22; C08G 63/78; C09D 4/00; C09D 135/02; C09D 167/04

(52) U.S. Cl. CPC C08G 63/60; A01N 25/10; A01N 31/16; A01P 1/00; A61F 2/30942; A61L 27/18; A61L 27/54; B33Y 10/00; B33Y 70/00; B33Y 80/00; C08F 22/20; C08F 22/22; C08G 63/78; C09D 4/00; C09D 135/02; C09D 167/04; A61F 2002/30062; A61F 2002/30092; A61F 2002/30677; A61F 2002/30985; A61L 2400/16; A61L 2430/02; C08G 2230/00

(57) ABSTRACT

The present invention provides novel polymeric materials for biomedical applications, derived from plant-derived phenolic diols, primarily zingerol. Two material classes are disclosed. First, biodegradable polyesters using said diols, are synthesized via solvent/catalyst-free melt polycondensation. These exhibit tunable mechanical properties, shape memory, antibacterial activity, and biocompatibility. Second, photopolymerizable zingerol derivative monomers or crosslinkers, are formulated into resins for 3D printing. Resulting 3D objects offer tailored mechanics, biodegradability, excellent shape memory, cytocompatibility, hemocompatibility, and potent antioxidant, antibacterial, and anti-biofilm functions. Methods for preparing both polymer types and their derivatives, alongside uses in tissue engineering and medical implants, are described.

### CROSS REFERENCE TO RELATED APPLICATION

This application claims the benefit of priority of U.S. Provisional Patent Application No. 63/650,546, filed May 22, 2024, the contents of which are all incorporated herein by reference in their entirety.

### TECHNICAL FIELD OF THE INVENTION

The present invention relates generally to novel polymeric materials derived from certain plant-derived phenolic diols, particularly zingerol which is a compound obtainable from ginger. More specifically, the invention pertains to biodegradable polyesters based on said plant-derived phenolic diols and photopolymerizable resin compositions based on zingerol and its derivatives, methods for their synthesis, and their applications, particularly in the biomedical field, including for tissue engineering, vascular grafts, nanoparticles, medical implants, and as 3D-printable materials with functional properties.

### BACKGROUND OF THE INVENTION

The widespread production and use of conventional polymers, predominantly derived from fossil fuels, have led to significant environmental concerns, including the accumulation of non-biodegradable waste and associated ecological damage. Annual production of such materials exceeds 380 million tons, and common disposal methods like landfilling and incineration contribute to pollution and economic losses. This has spurred considerable research into developing biodegradable polymers from renewable resources as a more sustainable alternative, aiming to mitigate environmental impact and foster a circular economy. Plants, as abundant and renewable bioresources, offer a rich platform for discovering new biodegradable polymeric materials. Among these, phenolic compounds derived from plants like ginger present unique structural features suitable for polymer synthesis. However, many biobased materials remain underexplored for synthesizing novel polymers with multifunctional properties.

In the biomedical field, there is a persistent and growing demand for advanced materials suitable for applications such as tissue engineering, medical implants, and drug delivery. The goal of tissue engineering is to repair or replace damaged tissues and organs, often relying on scaffolds that support cell growth and tissue regeneration. Ideally, these biomaterials should be non-toxic, biodegradable at an appropriate rate, possess adequate porosity, and exhibit mechanical properties that mimic the host tissue. While various materials like ceramics, metals, and composites have been used, biodegradable polymers are often favoured due to their tuneable synthesis, adaptable mechanical properties, adjustable biodegradability, and surface characteristics. Polyesters, in particular, have emerged as a significant class of biodegradable polymers for these applications. However, many commercially available biodegradable polymers, such as polycaprolactone (PCL), poly(glycolic acid) (PGA), and poly(lactic acid) (PLA), have limitations, including very long degradation times that may not be optimal for in-vivo tissue regeneration and can hinder clinical applicability.

Furthermore, medical implants, whether for orthopaedic, dental, or other applications, face several challenges. These include issues related to the host's reaction to the implant, the development of microbial biofilms on implant surfaces leading to recurrent infections, and subsequent inflammation around the implant site. Such complications are major causes of implant rejection and may necessitate repeated surgical interventions. Consequently, there is a long-felt need for implant materials with inherent antibacterial, anti-biofilm, and anti-inflammatory properties to improve implant success rates and patient outcomes. Reactive oxygen species (ROS) overproduction, for example, plays a significant role in peri-implantitis and other inflammatory responses, highlighting the need for materials with antioxidant capabilities.

The advent of three-dimensional (3D) printing technology has revolutionized the approach to fabricating medical devices and implants. This technology allows for the creation of intricate, customized, and patient-specific structures, from drug delivery systems to complex scaffolds that mimic anatomical forms. 3D printing offers high flexibility in design and material composition, enabling control over microstructure and potentially reducing production costs and times. While 3D printing holds immense promise, its full potential in the biomedical field is contingent upon the availability of suitable printable materials that are not only biocompatible and biodegradable but also possess the desired mechanical strength and functional properties.

Nature-derived compounds are increasingly being investigated as sources for new biomaterials due to their inherent biocompatibility and often beneficial biological activities. Ginger (Zingiber officinale) and its phenolic components, such as zingerone (the precursor to zingerol), zingerol itself, and other structurally related phenolic diols found in or derivable from ginger (including, for example, gingerols, and the reduced diol forms of shogaols and paradols where applicable), and similar plants, possess molecular structures featuring reactive hydroxyl groups and often inherent biological activities. These compounds have demonstrated a range of promising properties, including antibacterial, antifungal, anti-inflammatory, anti-diabetic, wound healing, anti-cancer, antioxidant, and anti-quorum sensing activities. Zingerone, for instance, is recognized by the U.S. Food and Drug Administration as “Generally Recognized as Safe” (GRAS). These intrinsic properties make zingerol, and by extension, other structurally analogous plant-derived phenolic diols from the ginger family and related sources, attractive candidates as building blocks for novel functional biomaterials.

Beyond material properties, the methods of polymer synthesis are also crucial. Many conventional polymer synthesis routes involve the use of potentially toxic catalysts or large volumes of organic solvents, which can raise manufacturing costs, pose environmental concerns, and complicate purification processes for biomedical applications. Thus, there is a motivation to develop simpler, more efficient, and environmentally friendly synthesis techniques, such as solvent-free and catalyst-free melt polycondensation.

Additionally, for many biomedical applications, particularly those involving minimally invasive surgery (MIS), materials with shape memory properties are highly desirable. Shape memory polymers (SMPs) can be temporarily deformed into a compact shape for easier insertion into the body via small incisions and then recover their original, functional shape upon exposure to a stimulus like body temperature. This capability can enhance surgical procedures, reduce patient trauma, and improve the functionality of implanted devices.

Therefore, there exists a significant and long-felt need in the art for novel polymeric materials that address the aforementioned limitations. Specifically, there is a need for:
(1) New biodegradable and biocompatible polymers, particularly those derived from readily available, safe, and functional natural resources like zingerol and the aforementioned related plant-derived phenolic diols (such as gingerols, and reduced forms of shogaols and paradols).
(2) Polymers whose thermal, mechanical, and degradation properties can be tuned to match specific biomedical applications, including mimicking various human tissues.
(3) Materials with inherent beneficial biological activities, such as antibacterial, anti-biofilm, anti-inflammatory, and antioxidant properties, to improve the success of medical implants and tissue engineering constructs.
(4) Environmentally friendly and cost-effective synthesis methods for these polymers, avoiding the use of harsh solvents or catalysts where possible.
(5) Novel photopolymerizable resin compositions, also based on functional natural building blocks like zingerol (or potentially other suitable plant-derived phenolic diols from the ginger family that can be similarly derivatized), that are suitable for 3D printable high-resolution, complex, patient-specific medical devices and scaffolds.
(6) 3D-printable materials that retain or enhance the beneficial biological activities of their core components and exhibit desirable characteristics such as appropriate mechanical strength, biodegradability, and shape memory behaviour.

The present invention aims to address these needs by providing novel biodegradable polyesters based on said class of plant-derived phenolic diols, with zingerol being a prime example, and 3D-printable photopolymerizable compositions derived primarily from zingerol, along with methods for their preparation and their use in various applications, particularly in the biomedical field.

### SUMMARY OF THE INVENTION

The present invention relates to biodegradable polyester polymers derived from certain plant-derived phenolic diols, methods of their preparation, and their uses, as well as 3D-printable zingerol-derivative compositions and methods. In one aspect, the invention provides a biodegradable polyester polymer comprises at least one chain of at least one diol linked via an ester bond to at least one diester, wherein said at least one diol and said at least one diester alternate along the chain and optionally cross-linked, and at least one terminal end of said polymer is a hydroxyl group.

In certain embodiments of such a polymer, said at least one chain may have Formula (I):

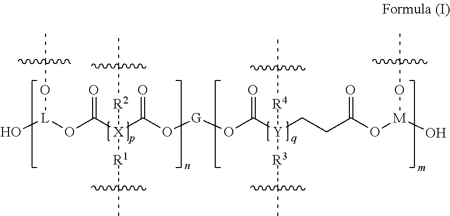

wherein —O-G-O— represents said diol,
X is a methylene or methylidene group of a polycarboxylic acid, said methylene or methylidene group is optionally substituted with R¹ and/or R² group, each independently selected from the group consisting of hydroxyl, carboxyl, carboxylate, (C₁-C₁₀)-alkanol, (C₁-C₁₀)-alkyl-carboxyl, and (C₁-C₁₀)-alkyl-carboxylate,
Y is a methylene or methylidene group of a polycarboxylic acid, said methylene or methylidene group is optionally substituted with R³ and/or R⁴ group, each independently selected from the group consisting of hydroxyl, carboxyl, carboxylate, (C₁-C₁₀)-alkanol, (C₁-C₁₀)-alkyl-carboxyl, and (C₁-C₁₀)-alkyl-carboxylate,
L and M are the same or different polyol moieties linked via an ester bond to said groups X and Y, respectively, through one of their hydroxyl groups,
n and m are integers independently selected from 1 to 10,000, and
p and q are integers independently selected from 1 to 24;
wherein said at least one chain is optionally cross-linked via an ester bond to at least one other chain through hydroxyl groups of said polyol moieties L and/or M, and/or through groups R¹, R², R³ and/or R⁴ of groups X and Y, respectively, and
wherein ‘ [symbol] ’ denotes an optional covalent bond.

The diol, G, in the aforementioned polymer structures may be a plant-derived diol. Such plant-derived diols can be selected from the group consisting of gingerols (such as 6-gingerol, 8-gingerol, and 10-gingerol); zingerol; and the diol forms obtained from the reduction of ketone-containing precursors like zingerone, shogaones (e.g., 6-shogaone, 8-shogaone, 10-shogaone, leading to their respective shogaol-diols), and paradones (leading to their respective paradol-diols). Zingerol itself is a reduced form of zingerone and may be derived from zingerone by reduction, for example, using NaBH₄. Specific examples of gingerols include 4-gingerol, 6-gingerol, 8-gingerol, or 10-gingerol. Specific examples of shogaols include 6-shogaol, 8-shogaol, or 10-shogaol.

In further embodiments of the polymers described, at least one of the polyol moieties L and M may be the same as the diol G. Alternatively, or in addition, the polyol moieties L and M may be sugar polyols, independently selected from materials such as xylitol, sorbitol, mannitol, maltitol, erythritol, lactitol, and isomalt. Xylitol, for instance, can be used as a crosslinker in these polymers.

The moieties [X]ₚ and [Y]_{q} in the polymer structures represent an alkyl or alkylene moiety of a diester of a polycarboxylic acid. This polycarboxylic acid can be independently selected from saturated and unsaturated, aliphatic, and aromatic polycarboxylic acids.

For example, the polycarboxylic acid may be a saturated aliphatic dicarboxylic acid selected from the group including oxalic acid, malonic acid, succinic acid, glutaric acid, adipic acid, pimelic acid, suberic acid, azelaic acid, sebacic acid (which can be used as a monomer), undecanedioic acid, dodecanedioic acid, brassylic acid, thapsic acid, japanic, phellogenic acid, and equisetolic acid.

Alternatively, the polycarboxylic acid may be an unsaturated aliphatic dicarboxylic acid selected from the group including crocetin, maleic acid, fumaric acid, glutaconic acid, 2-decenedioic acid, traumatic acid, muconic acid, glutinic acid, citraconic acid, mesaconic acid, and itaconic acid. In some cases, the polycarboxylic acid is a saturated aliphatic dicarboxylic acid substituted with at least one hydroxyl group (as R¹, R², R³, or R⁴). Examples include tartronic acid, malic acid, tartaric acid, α-hydroxy-glutaric acid, and saccharic acid.

The polycarboxylic acid could also be an aromatic dicarboxylic acid, such as phthalic acid, isophthalic acid, terephthalic acid, diphenic acid, and 2,6-naphthalene-dicarboxylic acid. If at least one of R¹, R², R³, or R⁴ is a carboxylic acid group, then the polycarboxylic acid is a tricarboxylic acid. Examples include citric acid (which can be used as a monomer), isocitric acid, aconitic acid, propane-1,2,3-tricarboxylic acid, agaric acid, and trimesic acid.

The invention also provides a method for preparing the biodegradable polyester polymers described above. This method comprises polymerising a monomer diol of the formula:

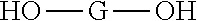

with at least one polycarboxylic acid of the formula:

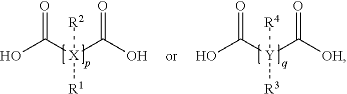

and
with at least one polyol of the formula:

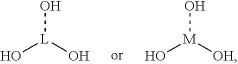

where X is a methylene or methylidene group of a polycarboxylic acid, said methylene or methylidene group is optionally substituted with R¹ and/or R² group, each independently selected from the group consisting of hydroxyl, carboxyl, carboxylate, (C₁-C₁₀)-alkanol, (C₁-C₁₀)-alkyl-carboxyl, and (C₁-C₁₀)-alkyl-carboxylate,
Y is a methylene or methylidene group of a polycarboxylic acid, said methylene or methylidene group is optionally substituted with R³ and/or R⁴ group, each independently selected from the group consisting of hydroxyl, carboxyl, carboxylate, (C₁-C₁₀)-alkanol, (C₁-C₁₀)-alkyl-carboxyl, and (C₁-C₁₀)-alkyl-carboxylate,
L and M are the same or different polyol moieties,
p and q are integers independently selected from 1 to 24; and
wherein ‘ [symbol] ’ denotes an optional covalent bond to an optional hydroxyl group of the polyol.

Preferably, this method is a catalyst-free and solvent-free multi-step poly-esterification synthesis, such as melt polycondensation. In a first step of such a synthesis, the reactant diol (e.g., zingerol), polycarboxylic acid (e.g., citric acid, sebacic acid), and optionally a polyol (e.g., xylitol) are introduced in a certain molar ratio into a reaction mixture. The reaction is carried out at a temperature typically ranging from about 60° C. to about 240° C., for instance, around 160° C. This reaction is usually carried out under continuous stirring and an inert atmosphere, such as nitrogen, for a period of about 0.5 to 3.5 hours, for instance, 1.5 hours, especially before the addition of a crosslinker if one is used separately.

In a subsequent step, a crosslinking agent, such as a polyol (e.g., xylitol), may be introduced or further reacted in the reaction mixture. This part of the reaction typically continues for an additional 0.5 to 3.5 hours, yielding an optionally cross-linked pre-polymer product. The molar ratios of the reactants (e.g., zingerol, sebacic acid, citric acid, xylitol) can be varied, for example, between 0.2 and 4.0 independently for each, to affect the physicochemical and mechanical properties of the obtained polymer products. In a final step of the synthesis, the pre-polymer product obtained is further cured (post-polymerization). This is often done in an oven at a temperature of about 80° C. to about 160° C. (e.g., 120° C.) for a period of about 2 to 8 consecutive days (e.g., 5 days) to complete the cross-linking.

The biodegradable polyester polymers of this invention are suitable for use in tissue engineering. The tissue targeted may be selected from human body tissues such as liver, uterus, bladder, and breast tissues, or specifically temporal or nasal cartilage. Furthermore, these polymers can be used in manufacturing shape-memory items, which may exhibit exceptional recovery responses at body temperature. They are also useful for manufacturing materials having antibacterial activity and for manufacturing implant items in general. The polymers also find use in regenerative medicine and wound healing, having demonstrated good in-vitro cytocompatibility and cell proliferation.

In another aspect, the invention provides photopolymerizable resin compositions for 3D printing. Such a composition comprises a zingerol derivative, wherein the zingerol is the diol as previously described (derived from ginger, being a reduced form of zingerone). This zingerol derivative comprises zingerol with at least one hydroxyl group thereof modified to incorporate a photopolymerizable group, where the photopolymerizable group is connected to the zingerol moiety via an ether, ester, or urethane linkage. Specific non-limiting examples of such zingerol derivatives include zingerol-glycidyl methacrylate (ZET), zingerol-methylacrylate (ZES), or zingerol-urethane (ZUR).

The invention further pertains to 3D-printed objects comprising a photopolymerized zingerol-derivative composition as just described. These objects are produced using a 3D printing process, such as DLP (Digital Light Processing), typically with the addition of about 1% photo-initiator. Such 3D-printed objects can exhibit a range of beneficial properties. These include tuneable thermal and mechanical characteristics, biodegradability, hemocompatibility (e.g., with haemolysis rates often less than 3-5%), shape memory efficacy, cytocompatibility with various human cell lines (such as HaCaT and BEAS-2B) and mouse fibroblast cells (NIH-3T3), anti-biofilm efficacy against bacteria like B. subtilis and E. coli, and/or antioxidant efficacy.

These 3D-printed objects can take the form of medical implants, scaffolds for tissue engineering (for example, bone tissue engineering), or devices for drug delivery, and are capable of being printed into high-resolution complex designs. In a particular embodiment, where the zingerol derivative is zingerol-glycidyl methacrylate (ZET), the resulting 3D-printed object (P-ZET) can exhibit shape memory properties with greater than 90% fixity and substantially 100% recovery (e.g., in approximately 2-4 seconds, depending on thickness). This makes them useful for minimally invasive implant applications, such as flexible nasal vestibular implants.

The invention also encompasses methods for preparing the zingerol derivatives suitable for the photopolymerizable resin compositions. Such a method comprises reacting zingerol (as previously defined) with a reagent capable of introducing a photopolymerizable (meth)acrylate group. This reaction results in the formation of an ether, ester, or urethane linkage connecting the zingerol moiety to the photopolymerizable group. For instance, a method for preparing a zingerol-glycidyl methacrylate (ZET) type derivative (having an ether linkage) involves reacting zingerol with glycidyl methacrylate (GMA), optionally in the presence of a base like triethyl-amine (TEA) and a solvent like ethyl acetate (EA), often under reflux conditions.

For preparing a zingerol-methylacrylate (ZES) type derivative (having an ester linkage), zingerol is reacted with methacrylic anhydride (MA), optionally in the presence of a catalyst like 4-dimethylaminopyridine (DMAP) and a solvent like ethyl acetate (EA). For preparing a zingerol-urethane (ZUR) type derivative (having a urethane linkage), zingerol is reacted with an isocyanatoalkyl methacrylate, such as isocyanatoethyl methacrylate (IEMA), optionally in the presence of a catalyst like stannous octoate (Sn(Oct)₂) and a solvent like 1,4-dioxane.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate embodiments of the invention and, together with the description, serve to explain the principles of the invention. The drawings are not necessarily to scale, emphasis instead being placed upon illustrating the principles of the invention.

FIG. 1 schematically represents the chemical reaction for the synthesis of zingerol (Zing-OH) by the reduction of zingerone.

FIGS. 2A and 2B demonstrate 1H NMR spectra for zingerol (FIG. 2A) and zingerone (FIG. 2B), respectively, which support the structural confirmation of these compounds.

FIG. 3A shows a thermogram from the thermogravimetric analysis (TGA) and derivative thermogravimetry (DTG) curves for zingerol, demonstrating its thermal stability and decomposition profile.

FIG. 3B shows a differential scanning calorimetry (DSC) curve for zingerol, from which its melting point can be determined.

FIG. 4 schematically illustrates the catalyst-free and solvent-free melt polycondensation reaction pathway for synthesizing ZCSX biodegradable polyesters, showing the involvement of zingerol, citric acid, sebacic acid, and xylitol.

FIG. 5 are digital snapshots of ZCSX (0.5), ZCSX (1.0), and ZCSX (1.5) before and after stretching.

FIG. 6A shows comparative FTIR spectra of zingerone, zingerol, and an exemplary ZCSX polyester, illustrating the spectral differences between them, thereby demonstrating the chemical changes occurring during zingerol synthesis and subsequent polyester formation.

FIG. 6B shows FTIR spectra of various ZCSX pre-polymer compositions prepared with different molar ratios of zingerol, highlighting spectral features before post-polymerization.

FIG. 6C shows FTIR spectra of post-cured ZCSX polyester compositions, illustrating further esterification and crosslinking upon curing by changes in characteristic peak intensities.

FIG. 7 shows ¹H NMR spectra for an exemplary ZCSX polyester, which supports the structural confirmation of this polymer.

FIG. 8A presents comparative TGA and DTG curves for different ZCSX poly-ester compositions, e.g., ZCSX (0.5), ZCSX (1.0), and ZCSX (1.5), illustrating their respective thermal stabilities and degradation patterns.

FIG. 8B presents DSC curves for different ZCSX polyester compositions, which demonstrate their glass transition temperatures T_{g} and amorphous or semi-crystalline nature.

FIG. 9A shows representative tensile stress-strain curves obtained from mechanical testing of different ZCSX polyester compositions, illustrating their distinct mechanical behaviours.

FIG. 9B is a bar graph that demonstrates a comparison of the elongation at break (EAB) values for different ZCSX polyester compositions, indicating their stretchability.

FIG. 9C is a bar graph that shows a comparison of the Young's modulus (YM) for different ZCSX polyester compositions, indicating their stiffness.

FIG. 9D is a bar graph that demonstrates a comparison of the ultimate tensile strength (UTS) for different ZCSX polyester compositions, indicating their tensile strength.

FIG. 9E is a bar graph that shows a comparison of the toughness, calculated from the stress-strain curves, for different ZCSX polyester compositions.

FIG. 9F is a graph that illustrates the in-vitro hydrolytic degradation behaviour of different ZCSX polyester compositions by showing the percentage weight loss over an extended period in a buffer solution.

FIG. 10A schematically illustrates the angles (θ_{f} and θᵣ) measured to determine the shape fixity and shape recovery ratios during the U-bending tests for assessing the shape memory properties of ZCSX polyesters.

FIG. 10B is a bar graph that shows a comparison of the recovery times required for different ZCSX polyester compositions to achieve 100% shape recovery.

FIG. 10C is a bar graph demonstrating a comparison of the shape fixity and shape recovery ratios for different ZCSX polyester compositions after a specified time.

FIG. 10D is a series of digital photographic images that sequentially document the shape recovery process of a ZCSX (1.5) polyester sample when placed in a 37° C. water bath after being deformed and frozen.

FIGS. 10E-10G are photographic images that illustrate the shape recovery capabilities of ZCSX polyesters from various complex temporary deformed shapes, such as helical (FIG. 10E), U-shaped helix (FIG. 10F), and coil (FIG. 10G) forms.

FIGS. 11A and 11B are graphs showing bacterial growth curves for E. coli and B. subtilis, respectively, which demonstrate the antibacterial effect of ZCSX polyesters in comparison to control conditions.

FIGS. 11C and 11D are photographic images of agar plates from colony formation assays that visually demonstrate the reduction in bacterial colonies of E. coli and B. subtilis, respectively, after exposure to ZCSX polyesters.

FIGS. 11E and 11F are Scanning Electron Microscopy (SEM) images that show the morphological changes and potential membrane damage of E. coli and B. subtilis bacteria, respectively, after interaction with ZCSX polyesters.

FIG. 12A is a bar graph that shows the viability of NIH/3T3 murine fibroblast cells after 1 and 3 days of exposure to conditioned media obtained from different ZCSX polyesters, demonstrating their cytocompatibility.

FIG. 12B presents a bar graph quantifying haemolysis percentages and corresponding photographic images of sample tubes, illustrating the hemocompatibility of ZCSX polyesters when incubated with human red blood cells.

FIGS. 12C and 12D are representative fluorescence microscopy images from live/dead staining assays of NIH/3T3 cells after 1 day and 3 days, respectively, of exposure to conditioned media from ZCSX polyesters, further demonstrating cytocompatibility.

FIGS. 12E and 12F are representative fluorescence microscopy images showing the morphology (F-actin filaments stained green and nuclei stained blue with DAPI) of NIH/3T3 cells after 1 day and 3 days, respectively, of exposure to conditioned media from ZCSX polyesters, illustrating cell health and structure.

FIGS. 13A-13C are representative microscopy images from an in-vitro scratch wound healing assay, showing the migration of NIH/3T3 fibroblast cells to close an artificial wound at 0 hrs. (FIG. 13A), 12 hrs. (FIG. 13B), 24 hrs. (FIG. 13C) when treated with conditioned media from ZCSX polyesters compared to a control.

FIG. 13D is a bar graph that quantitatively demonstrates the percentage of wound closure achieved at 12 and 24 hrs. for the conditions shown in FIGS. 13A-13C.

FIG. 14 shows the chemical reaction schemes for the synthesis of zingerol-based monomer compositions, namely zingerol-glycidyl methacrylate (ZET), zingerol-methylacrylate (ZES), and Zingerol-urethane (ZUR), from zingerol.

FIG. 15 schematically illustrates the photopolymerization mechanism where the (meth)acrylate groups of ZET, ZES, or ZUR compositions react to form a crosslinked polymer network upon exposure to light in the presence of a photoinitiator.

FIG. 16A presents FTIR spectra of the ZET, ZES, and ZUR photopolymerizable compositions, highlighting their characteristic absorption bands.

FIG. 16B presents ¹H NMR spectra for ZET, ZES, and ZUR, which are used for confirming their respective chemical structures.

FIGS. 16C and 16D shows the TGA/DTG curves and DSC thermograms, respectively, for the unpolymerized ZET, ZES, and ZUR compositions, demonstrating their thermal properties before polymerization.

FIGS. 16E and 16F shows the TGA/DTG curves and DSC thermograms for the 3D-printed (photopolymerized) materials P-ZET, P-ZES, and P-ZUR, demonstrating their thermal properties after polymerization.

FIGS. 17A-17C are photographs that show various examples of complex three-dimensional objects fabricated using ZET resin (FIG. 17A), ZES resin (FIG. 17B), and ZUR resin (FIG. 17C), demonstrating the printability of these compositions.

FIGS. 18A-18D show the comparative FTIR spectra of zingerol (FIG. 18A) and of ZET (FIG. 18B), ZES (FIG. 18C) and ZUR (FIG. 18D) before and after their photopolymerization, illustrating the disappearance of characteristic acrylate double bond peaks, thereby demonstrating successful polymerization.

FIG. 19A shows representative compressive stress-strain curves for the 3D-printed polymeric materials P-ZET, P-ZES, and P-ZUR, illustrating their mechanical response under compression.

FIG. 19B presents bar graphs that demonstrate a comparison of the ultimate compressive strength (UCS) and compressive stiffness (CS) for P-ZET, P-ZES, and P-ZUR.

FIG. 19C presents bar graphs that show a comparison of the strain at failure and toughness for P-ZET, P-ZES, and P-ZUR under compression.

FIG. 19D is a graph that illustrates the in-vitro hydrolytic degradation behaviour of P-ZET, P-ZES, and P-ZUR by showing the percentage weight loss over time in a buffer solution.

FIG. 19E is a bar graph that shows a comparison of shape fixity percentages and recovery times for the 3D-printed P-ZET, P-ZES, and P-ZUR materials, demonstrating their shape memory capabilities.

FIG. 19F is a graph that demonstrates the effect of varying print thickness on the shape fixity and recovery time for 3D-printed P-ZET.

FIG. 20A is a series of digital photographic images that sequentially document the shape memory recovery of a P-ZET strip (e.g., underwater at 37° C.) from a deformed state to its original shape.

FIG. 20B is a series of digital photographic images that sequentially document the shape memory recovery of a combined P-ZES and P-ZET 3D-printed object, such as a model nasal vestibular implant, at room temperature.

FIG. 21A presents bar graphs that show a comparison of cell viability after direct contact of NIH-3T3 murine fibroblast cells, HaCaT human skin cells, and BEAS-2B human lung cells with 3D-printed P-ZET, P-ZES, and P-ZUR materials at different time points.

FIG. 21B presents bar graphs that show a comparison of cell viability after exposure of NIH-3T3, HaCaT, and BEAS-2B cells to conditioned media obtained from P-ZET, P-ZES, and P-ZUR materials.

FIG. 22A presents a bar graph quantifying haemolysis percentages and corresponding photographic images of sample tubes, showing the hemocompatibility of 3D-printed P-ZET, P-ZES, and P-ZUR when incubated with human red blood cells, compared to positive and negative controls.

FIG. 22B presents UV-Vis spectra from a 3,3′,5,5′-tetramethylbenzidine (TMB) assay, along with corresponding photographic images of the solutions, demonstrating the ROS scavenging capability of P-ZET, P-ZES, and P-ZUR.

FIG. 22C is a bar graph that quantitatively demonstrates the ROS scavenging percentages for P-ZET, P-ZES, and P-ZUR based on the TMB assay.

FIGS. 23A and 23B are photographic images of agar plates from colony formation assays, visually demonstrating the antibacterial activity of P-ZET, P-ZES, and P-ZUR against B. subtilis and E. coli, respectively.

### DETAILED DESCRIPTION OF THE INVENTION

In the following description, various aspects of the present application will be described. For purposes of explanation, specific details are set forth to provide a thorough understanding of the present application. However, it will also be apparent to one skilled in the art that the present application may be practiced without the specific details presented herein. Furthermore, well-known features may be omitted or simplified in order not to obscure the present application.

The present invention relates to novel zingerol-based biodegradable polyesters and 3D-printable photopolymerizable compositions, methods for their preparation, and their uses, particularly in the biomedical field. Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The terms “comprising,” “comprised of,” “having,” “including,” and their conjugates, mean “including but not limited to.” These terms are open-ended and mean the elements recited, or their equivalent in structure or function, plus any other element or elements which are not recited. They should not be interpreted as being restricted to the means listed thereafter and do not exclude other elements or steps. Thus, the scope of an expression such as “a product comprised of x and z” should not be limited to products composed only of components x and z. Similarly, “a method comprising the steps x and z” should not be limited to methods consisting only of these steps.

The term “consisting of” means “including and limited to.” The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a,” “an,” and “the” include plural references unless the context clearly dictates otherwise. For example, the term “a compound” or “at least one compound” may include a plurality of compounds, including mixtures thereof.

Unless specifically stated, as used herein, the term “about” is understood as within a range of normal tolerance in the art, for example, within two standard deviations of the mean. In some embodiments, “about” means within 10% of the reported numerical value, preferably within 5%, and more preferably within 1%, 0.5%, 0.1%, 0.05%, or 0.01% of the stated value. In other embodiments, “about” can encompass a higher tolerance of variation depending on the experimental technique used or the context of the invention. Said variations of a specified value are understood by the skilled person and are within the context of the present invention. For example, a numerical range of “about 1 to about 5” should be interpreted to include not only the explicitly recited values from about 1 to about 5, but also individual values (e.g., 2, 3, 4) and sub-ranges (e.g., 1-3, 2-4, 3-5) within the indicated range. This principle also applies to ranges reciting only one numerical value as a minimum or a maximum. Unless otherwise clear from context, all numerical values provided herein are modified by the term “about”.

Other similar terms, such as “substantially,” “generally,” “up to,” and the like, are to be construed as modifying a term or value such that it is not an absolute. Such terms will be defined by the circumstances and the terms that they modify as those terms are understood by those skilled in the art. This includes, at very least, the degree of expected experimental error, technical error, and instrumental error for a given experiment, technique, or instrument used to measure a value.

As used herein, the term “and/or” includes any and all combinations of one or more of the associated listed items. It will be understood that when an element is referred to as being “on,” “attached to,” “connected to,” “coupled with,” “contacting,” etc., another element, it can be directly on, attached to, connected to, coupled with, or contacting the other element, or intervening elements may also be present. In contrast, when an element is referred to as being, for example, “directly on,” “directly attached to,” “directly connected to,” “directly coupled with,” or “directly contacting” another element, there are no intervening elements present. References to a structure or feature that is disposed “adjacent” to another feature may have portions that overlap or underlie the adjacent feature.

The term “subject” or “patient” as used herein refers to an animal, preferably a mammal, and most preferably a human, who is the object of treatment, observation, or experiment.

The term “biodegradable” refers to a material that can be broken down by the action of living organisms, typically microorganisms, or by natural chemical processes (e.g., hydrolysis) in a physiological environment, into simpler substances that are non-toxic or can be metabolized or excreted by the body. The term “biocompatible” refers to a material that does not elicit any undesirable local or systemic effects in a host, such as an immune response, toxicity, or inflammation, when in contact with the biological system of that host.

The term “diol” refers to an organic compound containing two hydroxyl (—OH) groups. The term “polyol” refers to an organic compound containing multiple hydroxyl groups (—OH). Diols, triols (three —OH groups), and tetrols (four —OH groups) are examples of polyols. The term “sugar polyol,” also known as sugar alcohol, polyhydric alcohol, polyalcohol, or alditol, refers to an organic compound, typically derived from sugars, containing more than one hydroxyl group attached to a corresponding carbon atom. Non-limiting examples of sugar polyols include xylitol, sorbitol, mannitol, maltitol, erythritol, lactitol, and isomalt. Xylitol is a preferred sugar polyol in some embodiments of the present invention.

In the context of the present invention, particularly for the synthesis of the biodegradable polyesters described herein, the term “plant-derived diol” encompasses not only zingerol but also other structurally related phenolic diols obtainable from ginger (Zingiber officinale) or similar natural sources. These include, but are not limited to, various gingerols (e.g., 6-gingerol, 8-gingerol, 10-gingerol, which possess suitable diol functionalities) and the reduced diol forms of related phenolic ketones such as shogaols (e.g., reduced 6-shogaol, 8-shogaol, 10-shogaol) and paradols (e.g., reduced paradol).

These compounds share a common structural heritage with zingerol, often featuring a substituted phenyl ring with at least one hydroxyl group and an aliphatic chain also containing at least one hydroxyl group, making them suitable candidates for polycondensation reactions to form polyesters. While zingerol is extensively exemplified, it is contemplated that these related plant-derived diols can be utilized in analogous synthetic procedures to yield polyesters with potentially similar or complementary advantageous biomedical properties due to their shared structural motifs and natural origin.

The term “polycarboxylic acid” refers to an organic compound containing two or more carboxyl functional groups (—COOH). Polycarboxylic acids containing two carboxyl groups are referred to as dicarboxylic acids, and those containing three carboxyl groups are referred to as tricarboxylic acids. Polycarboxylic acids can be saturated (having all single bonds between its carbon atoms) or unsaturated (having at least one double bond within the carbon-carbon chain).

Non-limiting examples of saturated aliphatic dicarboxylic acids include oxalic acid, malonic acid, succinic acid, glutaric acid, adipic acid, pimelic acid, suberic acid, azelaic acid, sebacic acid, undecanedioic acid, dodecanedioic acid, brassylic acid, thapsic acid, japanic, phellogenic acid, and equisetolic acid. Sebacic acid is a preferred saturated aliphatic dicarboxylic acid in some embodiments. Non-limiting examples of unsaturated aliphatic dicarboxylic acids include crocetin, maleic acid, fumaric acid, glutaconic acid, 2-decenedioic acid, traumatic acid, muconic acid, glutinic acid, citraconic acid, mesaconic acid, and itaconic acid.

Non-limiting examples of saturated aliphatic dicarboxylic acids substituted with at least one hydroxyl group include tartronic acid, malic acid, tartaric acid, α-hydroxy-glutaric acid, and saccharic acid. Non-limiting examples of aromatic dicarboxylic acids include phthalic acid, isophthalic acid, terephthalic acid, diphenic acid, and 2,6-naphthalenedicarboxylic acid. Non-limiting examples of saturated aliphatic tricarboxylic acids include citric acid, isocitric acid, aconitic acid, propane-1,2,3-tricarboxylic acid, agaric acid, and trimesic acid. Citric acid is a preferred tricarboxylic acid in some embodiments.

The term “methylene group” refers to any part of a molecule that consists of two hydrogen atoms bound to a carbon atom, which is connected to the remainder of the molecule by two single bonds (e.g., —CH₂— or >CH₂). The term “methylidene group” refers to any part of a molecule that consists of two hydrogen atoms bound to a carbon atom, which is connected to the remainder of the molecule by a double bond (e.g., ═CH₂).

“Zingerone” refers to 4-(4-hydroxy-3-methoxyphenyl)-2-butanone, which is a phenolic compound found in ginger. “Zingerol” or “Zing-OH” as used herein refers to 4-(4-hydroxy-3-methoxyphenyl)-2-butanol, which is the reduced form of zingerone, typically obtained by the reduction of the ketone group of zingerone to a hydroxyl group. Zingerol is a diol used as a primary monomer in embodiments of the present invention. In the context of this invention, plant-derived diols may include zingerol.

“Gingerols,” “shogaols,” and “paradol” are other phenolic compounds found in ginger, which, along with zingerol, can be considered as plant-derived diols or sources for derivatives thereof. Non-limiting examples of gingerols include 6-gingerol, 8-gingerol, and 10-gingerol; examples of shogaols include 6-shogaol, 8-shogaol, and 10-shogaol.

A “diester of a polycarboxylic acid” refers to a molecule formed when a polycarboxylic acid reacts with at least one diol to form ester bonds. In the context of the polyesters of the present invention, it generally refers to the polycarboxylic acid monomer unit within the polymer chain. A “polyester” is a polymer in which the monomer units are linked by ester chemical groups. In the present invention, it refers to polymers formed, for example, by the reaction of diols (like zingerol) with polycarboxylic acids (like sebacic acid and citric acid) and optionally other polyols (like xylitol). “ZCSX polyesters” refers to specific embodiments of biodegradable polyesters synthesized using zingerol (Zing-OH), citric acid (CA), sebacic acid (SA), and xylitol (Xy) as co-monomers and/or crosslinkers.

“Melt polycondensation” is a polymerization method where monomers react in their molten state to form a polymer, typically with the elimination of a small molecule like water, without the use of solvents. “Pre-polymer” refers to an oligomeric or partially polymerized material that can undergo further polymerization or crosslinking to form the final polymer.

“Post-polymerization” or “curing” refers to a process of further reaction of a pre-polymer to increase molecular weight, achieve crosslinking, and develop the final properties of the polymer material. “Crosslinked polymer” refers to a polymer in which polymer chains are interconnected by covalent bonds (crosslinks), forming a three-dimensional network structure.

“Photopolymerizable resin composition” refers to a liquid formulation that can be solidified or cured by exposure to light, typically ultraviolet (UV) or visible light, usually in the presence of a photoinitiator.

“Zingerol derivative” in the context of the 3D-printable compositions refers to a molecule derived from zingerol where at least one of the hydroxyl groups of zingerol has been chemically modified to incorporate a photopolymerizable group, such as a (meth)acrylate group. These derivatives can function as both monomers or crosslinkers during photopolymerization due to the potential for multiple photopolymerizable groups per molecule. This modification involves connecting the photopolymerizable group to the zingerol moiety via an ether, ester, or urethane linkage:
“ZET” or “zingerol-glycidyl methacrylate” refers to a zingerol derivative formed by reacting zingerol with glycidyl methacrylate (GMA), resulting in a molecule with methacrylate groups suitable for photopolymerization and an ether linkage.
“ZES” or “zingerol-methylacrylate” refers to a zingerol derivative formed by reacting zingerol with methacrylic anhydride (MA), resulting in a molecule with methacrylate groups suitable for photopolymerization and ester linkages.
“ZUR” or “zingerol-urethane” (also referred to as zingerol-isocyanate in some contexts of its precursor) refers to a zingerol derivative formed by reacting zingerol with an isocyanatoalkyl (meth)acrylate such as isocyanatoethyl methacrylate (IEMA), resulting in a molecule with methacrylate groups suitable for photopolymerization and urethane linkages.

“(Meth)acrylate group” refers to an acrylate (—O—C(═O)—CH═CH₂) or a methacrylate (—O—C(═O)—C(CH₃)═CH₂) functional group, which is a common photo-polymerizable moiety. “Photopolymerization” is a process of polymerization that is initiated by the absorption of light. “Photoinitiator” is a compound that, upon absorption of light, generates reactive species (e.g., free radicals or cations) that initiate polymerization. TPO (diphenyl(2,4,6-trimethylbenzoyl)phosphine oxide) is an example of a photoinitiator used in some embodiments.

“3D Printing” refers to additive manufacturing processes used to create three-dimensional objects from a digital model by successively adding material layer by layer. “DLP (Digital Light Processing) 3D Printing” is a specific type of 3D printing that uses a digital light projector to cure layers of photopolymer resin.

“Shape memory” refers to the ability of a material to recover its original, permanent shape from a significant, quasi-plastic temporary deformation upon application of an external stimulus such as heat, light, or a change in pH or solvent. “Shape fixity ratio” quantifies the ability of a material to maintain its temporary deformed shape after the removal of the deformation force and cooling/setting. “Shape recovery ratio” quantifies the extent to which a material recovers its original permanent shape after application of the appropriate stimulus.

“Cytocompatibility” refers to the ability of a material to be in contact with cells without eliciting any detrimental local or systemic effects, such as toxicity or adverse alterations in cellular behavior. A cytocompatible material is one that is compatible with cells, meaning it is essentially non-toxic and supports or does not significantly impede normal cellular functions, including viability, proliferation, and morphology. Assessment of cytocompatibility is often performed through in-vitro studies that measure these cellular responses. “Hemocompatibility” refers to the property of a material being compatible with blood, meaning it does not cause significant adverse reactions such as haemolysis (rupturing of red blood cells) or thrombosis when in contact with blood. A haemolysis rate of less than 5% is generally considered hemocompatible.

“Antibacterial activity” refers to the ability of a material to kill bacteria or inhibit their growth or proliferation. “Anti-biofilm efficacy” refers to the ability of a material to prevent the formation of biofilms (communities of microorganisms adhered to a surface and encased in an extracellular polymeric matrix) or to disrupt existing biofilms. “Antioxidant efficacy” or “ROS scavenging” refers to the ability of a material to neutralize reactive oxygen species (ROS) or other free radicals, thereby reducing oxidative stress and potential damage to cells and tissues.

“Mechanical properties” refer to the characteristics of a material that describe its response to applied forces, including but not limited to:
“Ultimate Tensile Strength (UTS)”: The maximum stress a material can withstand while being stretched or pulled before necking.
“Elongation at Break (EAB)”: The ratio between changed length and initial length after breakage of the test specimen.
“Young's Modulus (YM)” or “Modulus of Elasticity”: A measure of the stiffness of a solid material.
“Toughness”: The ability of a material to absorb energy and plastically deform before fracturing.
“Ultimate Compressive Strength (UCS)”: The maximum compressive stress a material can withstand before failure.
“Compressive Stiffness (CS)” or “Compressive Modulus”: A measure of a material's resistance to elastic deformation under compression.

### Zingerol-Based Biodegradable Polyesters

In one major aspect, the present invention provides novel biodegradable polyester polymers. These polymers, in certain embodiments, are primarily derived from zingerol, a compound obtainable from the renewable resource ginger, and are notably synthesized using an environmentally considerate solvent-free and catalyst-free melt polycondensation process.

The polyesters according to this aspect of the invention are generally characterized by polymer chains wherein plant-derived diol units, particularly as exemplified by zingerol, are linked via ester bonds to diester units derived from polycarboxylic acids, with these units alternating along the chain. Furthermore, these polymer chains may be optionally cross-linked, typically through the use of multifunctional monomers, and the resulting polymers characteristically possess at least one terminal hydroxyl group.

### Synthesis and Characterization of Zingerol

The foundation of these novel polyesters, in many preferred embodiments, lies in the utilization of zingerol as a principal diol monomer. Zingerol, chemically identified as 4-(4-hydroxy-3-methoxyphenyl)-2-butanol, offers unique structural features derived from its parent compound, zingerone, which is naturally found in ginger. In an exemplary embodiment related to the preparation of this key monomer, zingerol is synthesized by the chemical reduction of zingerone.

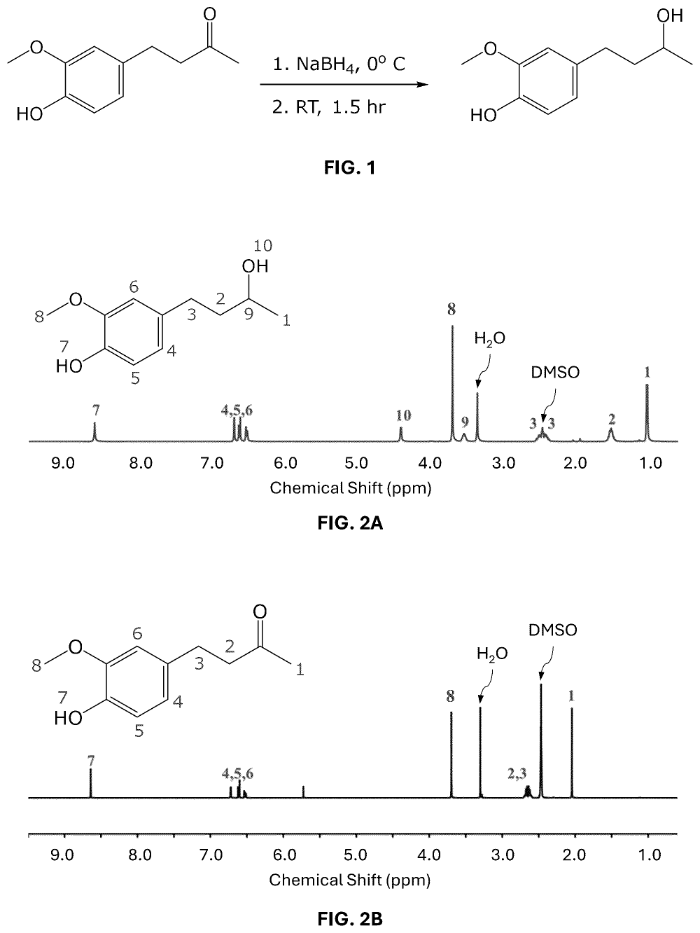

This synthetic transformation is schematically represented in FIG. 1. The process, in a specific embodiment, involves dissolving zingerone (for instance, 1 molar equivalent based on its molecular weight of 194.23 g/mol) in a suitable solvent, typically an alcohol such as methanol, for example, using about 3 volumes of solvent relative to the mass of zingerone. A reducing agent, commonly sodium borohydride is then introduced to conduct the reduction of the ketone group in zingerone to a secondary alcohol group in zingerol. Sodium borohydride may be used in a quantity such as about 0.75 to 0.80 molar equivalents (e.g., about 0.02 mol for 0.025 mol of zingerone), and its addition is preferably performed portion-wise or gradually to the zingerone solution, often while maintaining the reaction mixture at a reduced temperature, for instance, approximately 0° C. using an ice bath, to effectively manage the reaction's exothermicity and selectivity.

Following the complete addition of the reducing agent, the reaction mixture is typically stirred at ambient room temperature (e.g., 20-25° C.) for a duration sufficient to ensure the reaction proceeds to completion, which may be in the order of about 1.0 to 2.0 hours, for example, approximately 1.5 hours. The progress of this reduction reaction can be conveniently monitored using standard chromatographic techniques, such as TLC, by observing the disappearance of the zingerone spot and the appearance of the zingerol spot.

Upon completion of the reduction reaction, the zingerol product is isolated and purified. An exemplary workup procedure involves quenching any excess reducing agent, followed by partitioning the product between an aqueous phase and an organic extraction solvent, such as ethyl acetate. The organic layers containing the product are combined, washed (for instance, with brine to remove water-soluble impurities), and then dried over a suitable anhydrous drying agent, such as sodium sulphate. Finally, the organic solvent is removed, typically by rotary evaporation under reduced pressure, to yield zingerol, often as a viscous liquid or low-melting solid.

The successful synthesis and chemical structure of zingerol are thoroughly confirmed by various analytical techniques. For example, a comparative FTIR spectra of zingerol and zingerone shows the disappearance of the strong ketone carbonyl (C═O) stretching peak characteristic of zingerone (e.g., around 1702 cm⁻¹) and the appearance or enhancement of hydroxyl (—OH) group absorption bands (e.g., broad peak in the 3200-3600 cm⁻¹ region) confirm the reduction.

Further structural confirmation is provided by ¹H NMR spectroscopy, as exemplified in FIGS. 2A and 2B, where the ¹H NMR spectrum of zingerol and zingerone, respectively, shows characteristic signals, including, for example, a new peak (9) for the methylene proton of the group —CH(OH)— (around 3.65 ppm) of zingerol, a new peak (10) for the hydroxyl proton of the —CH(OH)— group (around 4.12 ppm) of zingerol, and the upfield shift in the signals of adjacent methyl (1) and methylene (2) protons of the product zingerol compared to zingerone, confirming the formation of the secondary alcohol.

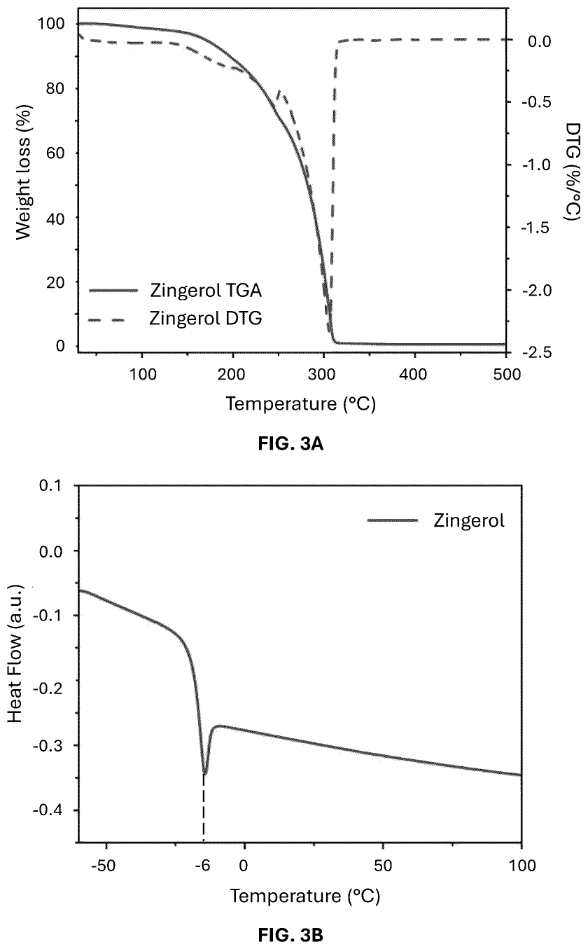

Thermal characteristics of the synthesized zingerol, such as its thermal stability profile via thermogravimetric analysis (TGA) and its melting point via differential scanning calorimetry (DSC), are illustrated in FIGS. 3A-3B, respectively. The TGA thermogram (FIG. 3A) shows that zingerol is stable up to about 165° C., with its main decomposition occurring at higher temperatures (e.g., maximum weight loss around 280° C.). The DSC graph (FIG. 3B) indicates a melting point for zingerol at −6° C., that is consistent with its often liquid or semi-solid appearance at room temperature.

### Synthesis and Characterization of ZCSX Biodegradable Polyesters

In further aspects and embodiments, the present invention provides biodegradable polyesters having the general structure of Formula (I):

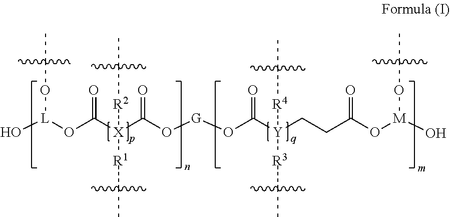

wherein —O-G-O— represents a plant-derived diol moiety G,
X is a methylene or methylidene group of a polycarboxylic acid, said methylene or methylidene group is optionally substituted with R¹ and/or R² group, each independently selected from the group consisting of hydroxyl, carboxyl, carboxylate, (C₁-C₁₀)-alkanol, (C₁-C₁₀)-alkyl-carboxyl, and (C₁-C₁₀)-alkyl-carboxylate,
Y is a methylene or methylidene group of a polycarboxylic acid, said methylene or methylidene group is optionally substituted with R³ and/or R⁴ group, each independently selected from the group consisting of hydroxyl, carboxyl, carboxylate, (C₁-C₁₀)-alkanol, (C₁-C₁₀)-alkyl-carboxyl, and (C₁-C₁₀)-alkyl-carboxylate,
L and M are the same or different polyol moieties linked via an ester bond to said groups X and Y, respectively, through one of their hydroxyl groups,
n and m are integers independently selected from 1 to 10,000, and
p and q are integers independently selected from 1 to 24;
wherein said at least one chain is optionally cross-linked via an ester bond to at least one other chain through hydroxyl groups of said polyol moieties Land/or M, and/or through groups R¹, R², R³ and/or R⁴ of groups X and Y, respectively, and
   wherein ‘ [symbol] ’ denotes an optional covalent bond.

In preferred embodiments, as exemplified by the ZCSX series, this moiety G is derived from zingerol. However, it is an aspect of this invention that G can also be derived from other plant-derived diols possessing suitable reactivity for poly-esterification, particularly those structurally related to zingerol. Such plant-derived diols G can be selected from the group consisting of gingerols (e.g., 6-gingerol, 8-gingerol, 10-gingerol), zingerol itself, and the reduced diol forms of paradol, zingerone (which is zingerol), and shogaols.

It is contemplated by the inventors that for ketone-containing precursors within this group, such as zingerone itself or the corresponding ketone precursors of various shogaols and paradols, a ketone-to-alcohol reduction process, similar to that successfully demonstrated herein for the synthesis of zingerol from zingerone, as shown in FIG. 1, is applicable for the synthesis of their corresponding diols suitable for polyesterification.

Furthermore, these related phenolic compounds derived from ginger and similar plant sources often share common biosynthetic origins and chemical interrelationships; for instance, shogaols can be formed from gingerols via dehydration, and zingerone can be formed from gingerols via retro-aldol reactions, highlighting their structural and chemical kinship. These diols typically possess at least two hydroxyl groups capable of participating in esterification reactions with polycarboxylic acids.

The structural similarities among these compounds, often featuring phenolic groups and aliphatic hydroxyls on a related phenylpropanoid-derived skeleton, suggest that they can be incorporated into polyester backbones using synthetic methods analogous to those described herein for zingerol. The incorporation of these different plant-derived diols into the polyester structure of Formula (I) is expected to yield materials with variations in properties (e.g., flexibility, degradation rate, specific biological activities) while retaining the core advantages of biodegradability and biocompatibility inherent to such natural precursors.

These polyesters are prepared by co-polymerizing said plant-derived diol (G) with other monomers, preferably derived from renewable resources. Such co-monomers typically include one or more polycarboxylic acids, for instance, a dicarboxylic acid such as sebacic acid (SA), and a tricarboxylic acid such as citric acid (CA) which can also promote crosslinking. Additionally, other polyols, such as the sugar alcohol xylitol, may be incorporated, often serving as a crosslinking agent due to its multiple hydroxyl groups.

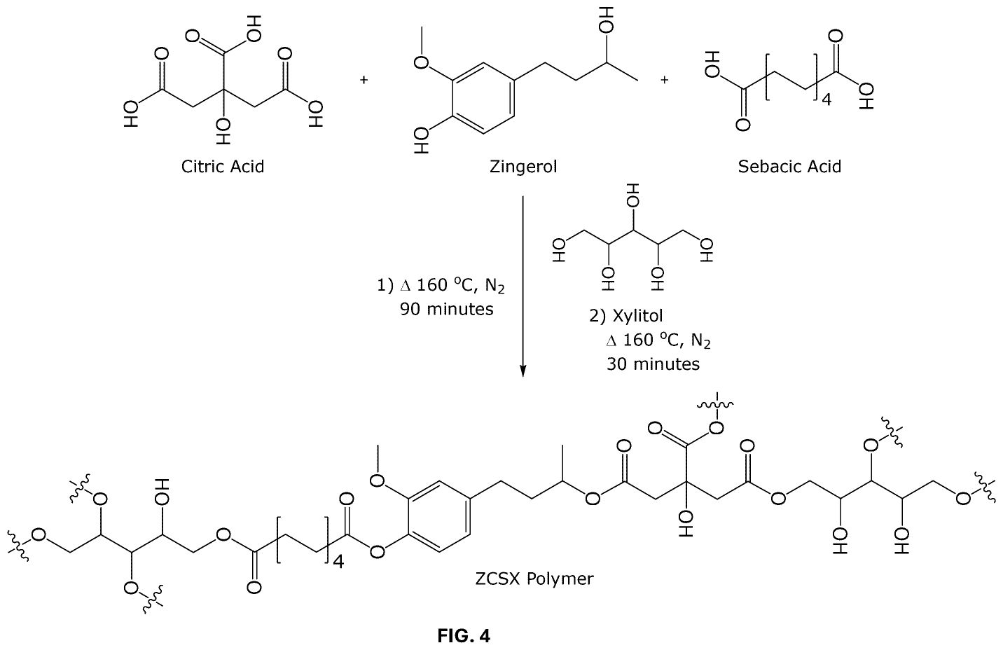

A significant and advantageous feature of the synthesis method disclosed in the present invention is its solvent-free and catalyst-free nature, employing a melt polycondensation technique, as schematically illustrated in FIG. 4 for the non-limiting example of the ZCSX polyester synthesis. This environmentally benign approach simplifies the process and minimizes concerns about residual toxic materials in the final polymer, which is particularly important for biomedical applications.

The invention provides a method for preparing the biodegradable polyester polymers described above. This method comprises polymerising a monomer diol of the formula:

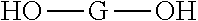

with at least one polycarboxylic acid of the formula:

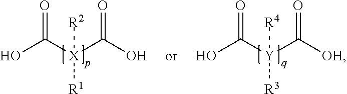

and
with at least one polyol of the formula:

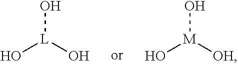

where X is a methylene or methylidene group of a polycarboxylic acid, said methylene or methylidene group is optionally substituted with R¹ and/or R² group, each independently selected from the group consisting of hydroxyl, carboxyl, carboxylate, (C₁-C₁₀)-alkanol, (C₁-C₁₀)-alkyl-carboxyl, and (C₁-C₁₀)-alkyl-carboxylate,
Y is a methylene or methylidene group of a polycarboxylic acid, said methylene or methylidene group is optionally substituted with R³ and/or R⁴ group, each independently selected from the group consisting of hydroxyl, carboxyl, carboxylate, (C₁-C₁₀)-alkanol, (C₁-C₁₀)-alkyl-carboxyl, and (C₁-C₁₀)-alkyl-carboxylate,
L and M are the same or different polyol moieties,
p and q are integers independently selected from 1 to 24; and
wherein ‘ [symbol] ’ denotes an optional covalent bond to an optional hydroxyl group of the polyol.

The synthesis of these ZCSX polyesters, in a preferred embodiment, is conducted in two primary stages: an initial pre-polymerization stage to form an oligomeric pre-polymer, followed by a post-polymerization or curing stage to achieve the final, typically crosslinked, polyester material. In the pre-polymerization stage, the selected plant-derived diol G (e.g., zingerol in specific embodiments like ZCSX), sebacic acid (SA), and citric acid (CA) are combined in specific molar ratios in a suitable reaction vessel. For instance, as detailed in Table 1 below, various ZCSX formulations specifically made with zingerol can be prepared by varying the molar ratio of zingerol (e.g., ZCSX (0.5), ZCSX (1.0), ZCSX (1.5) corresponding to 0.5, 1.0, and 1.5 molar equivalents of zingerol, respectively, relative to fixed amounts of sebacic acid (e.g., 1 molar equivalent) and citric acid (e.g., 0.5 molar equivalents)). Xylitol, if used as an additional crosslinker, is also added in a defined molar ratio (e.g., 0.5 molar equivalents).

TABLE 1
Molar ratio of zingerol, citric acid, sebacic acid,
and xylitol for synthesis of different ZCSX polyesters
and their corresponding post-polymer appearance.

| | ZCSX | ZCSX | ZCSX | ZCSX |
|---|---|---|---|---|
| Monomer | (0.5) | (1.0) | (1.5) | (2.0) |
| Zingerol | 0.5 | 1 | 1.5 | 2.0 |
| Sebacic Acid | 1 | 1 | 1 | 1 |
| Citric Acid | 0.5 | 0.5 | 0.5 | 0.5 |
| Xylitol | 0.5 | 0.5 | 0.5 | 0.5 |
| Nature of the | Soft-tissue | Soft-tissue | Adhesive & soft- | Incomplete |
| post-polymer | like | like | tissue like, and | cross- |
| | elastomeric | stretchable | highly stretchable | linking |

The mixture of these monomers is then heated to a temperature, typically in the range of about 150° C. to about 170° C., for example, about 160° C., that allows for melt polycondensation to occur. This reaction is carried out under inert atmosphere, such as a continuous purge of nitrogen gas, and with constant mechanical stirring to ensure homogeneity and facilitate the removal of condensation byproducts (e.g., water). The pre-polymerization reaction is allowed to proceed for a specific duration, for example, about 1.5 hours for the initial reaction of zingerol, SA, and CA, after which xylitol may be introduced, and the reaction continued for an additional period, such as about 0.5 hours, to ensure its incorporation. The resulting pre-polymer is typically a viscous, waxy, brownish material, which is generally soluble in various organic solvents like chloroform, THF, acetone, methanol, and ethanol, indicating it is not yet fully crosslinked.

Following the formation of the pre-polymer, the second stage involves post-polymerization or curing. The pre-polymer material is transferred into a suitable mould, such as a Teflon-coated petri dish, to define its shape, and then subjected to heating in an oven at a specific temperature for an extended period. The curing temperature is typically somewhat lower than the pre-polymerization temperature, for example, in the range of about 100° C. to 130° C., such as 120° C. The curing duration is generally several days, for instance, about 5 consecutive days. During this curing stage, further esterification reactions occur between any remaining free hydroxyl and carboxyl groups, leading to an increase in molecular weight and the formation of a more extensively crosslinked three-dimensional polymer network.

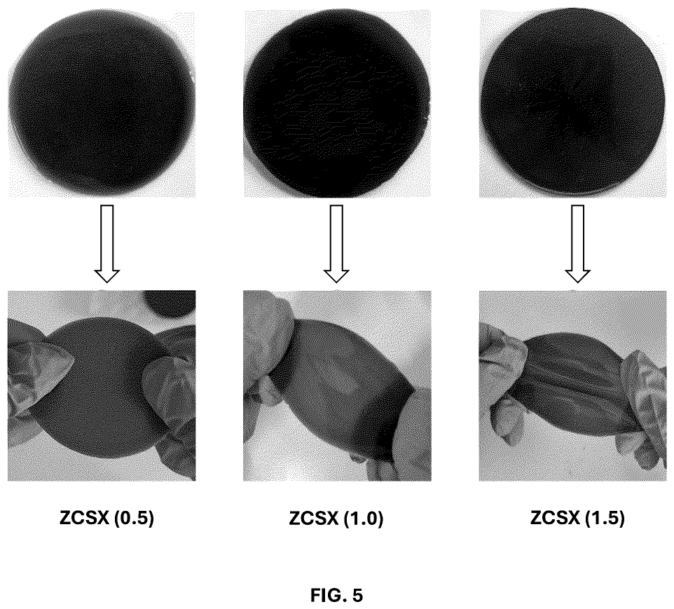

The physical state and properties of the final post-cured ZCSX polyester are significantly influenced by the initial monomer feed ratios, particularly the zingerol content and the overall balance of hydroxyl to carboxyl groups. This can result in materials ranging from soft, stretchable elastomers to more rigid structures as summarised in Table 1 above and visually indicated in FIG. 5 by the appearance and comparative behaviour of ZCSX (0.5), ZCSX (1.0), and ZCSX (1.5) samples, for example, before and after stretching. Formulations with a significant excess of zingerol, for example, ZCSX (2.0), might lead to incomplete crosslinking, resulting in materials that are adhesive and lack structural integrity.

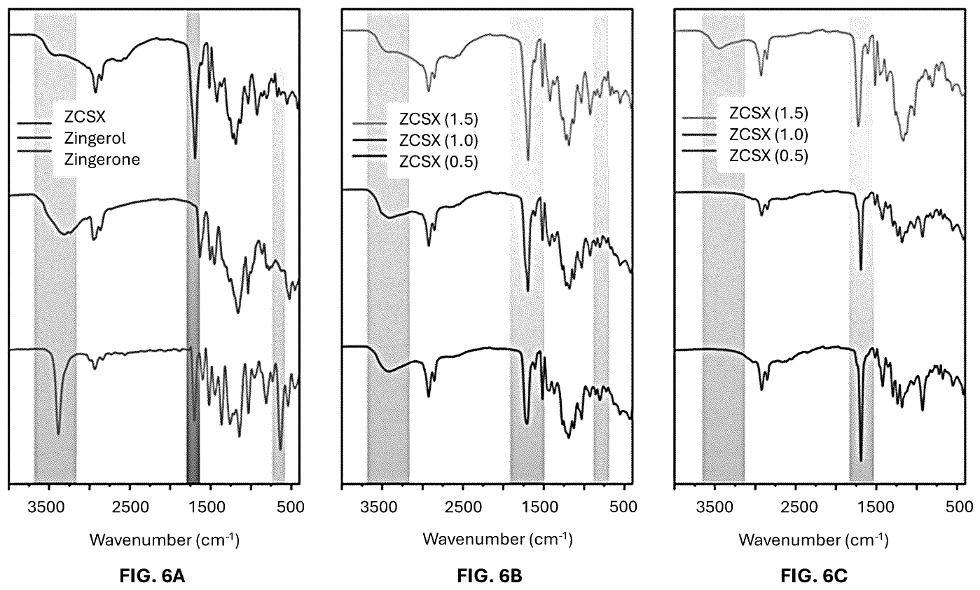

The chemical structure of the ZCSX polyesters and the success of the polymerization process are further confirmed by spectroscopic analysis. Reference is made to FIG. 6A providing a comparative FTIR spectrum, where the ZCSX polyester spectrum shows a prominent ester carbonyl (C═O) band at 1705 cm 1, indicative of polyester formation, along with characteristic peaks from the incorporated zingerol moiety (e.g., aromatic C—H bending around 750 cm⁻¹). FIG. 6B shows the FTIR spectra of ZCSX pre-polymers with different zingerol contents. In these pre-polymers, a broad absorption band for hydroxyl groups (around 3100-3500 cm⁻¹) is typically observed, the intensity and breadth of which can correlate with the zingerol feed ratio and the number of unreacted hydroxyl groups. The ester carbonyl peak is also present.

Upon post-curing, as shown in the FTIR spectra of post-cured ZCSX poly-esters in FIG. 6C, there is often a noticeable increase in the relative intensity of the ester carbonyl peak and a significant reduction or disappearance of the broad hydroxyl peak, especially for formulations like ZCSX (0.5) and ZCSX (1.0), indicating that further esterification and crosslinking reactions have taken place during the curing process, consuming the available hydroxyl and carboxyl groups. For compositions with higher initial zingerol content, for example ZCSX (1.5), a residual hydroxyl peak might still be observable after curing, consistent with a higher number of free hydroxyl groups in its structure.

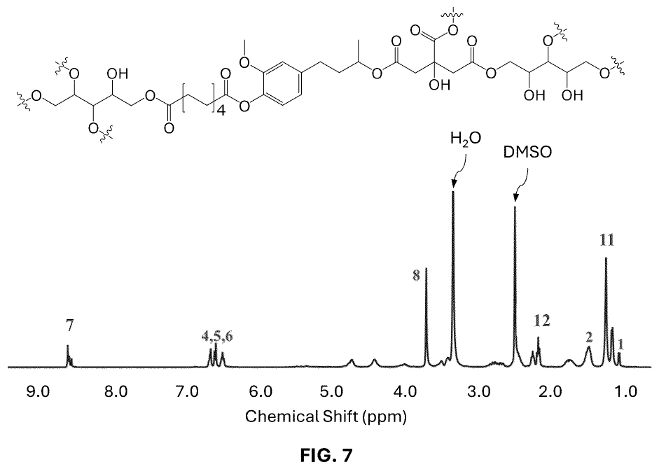

Further experimental support for the structural confirmation of the pre-polymer ZCSX is provided by 1H NMR spectroscopy, as exemplified in FIG. 7 compared to spectra in FIG. 2A of zingerol. While the spectrum of the polymeric material is complex due to many overlapping signals and broad peaks, characteristic signals corresponding to the protons of the incorporated zingerol, sebacic acid, citric acid, and xylitol monomer units can be identified, confirming their presence in the pre-polymer structure. For instance, signals from the aromatic protons (4, 5, 6) of zingerol around 6.6-6.7 ppm, aliphatic methylene protons (11) from the long chain of sebacic acid around 1.1 ppm, and those adjacent to ester linkages (12) around 2.3-2.4 ppm can be discerned.

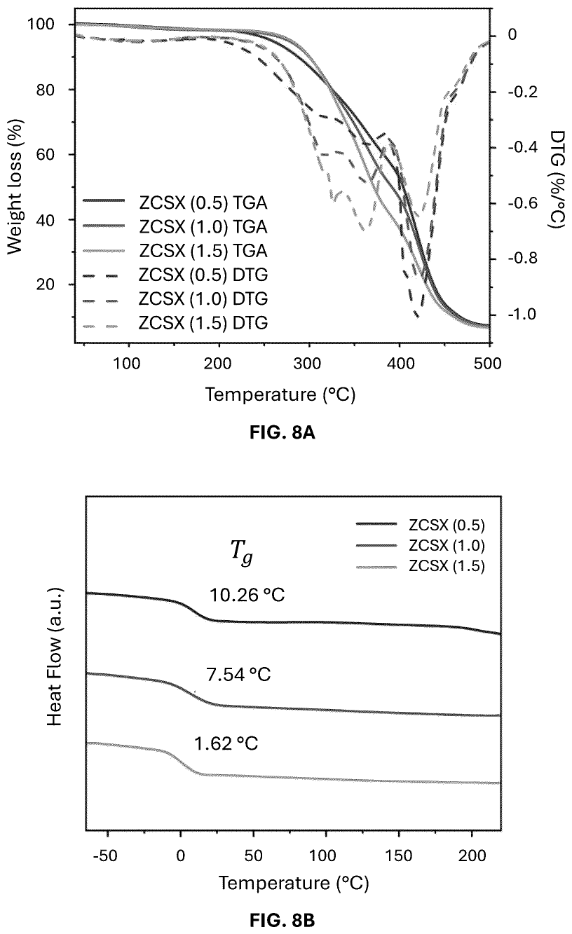

The thermal behaviour of these ZCSX polyesters is elucidated by TGA and also by DSC. As shown by TGA curves in FIG. 8A, the ZCSX polyesters generally exhibit good thermal stability, often being stable up to temperatures around 250° C. before significant decomposition begins. The thermal stability can be influenced by the zingerol content; for instance, an increase in zingerol concentration might lead to a slight decrease in thermal stability, possibly due to a lower crosslinking density if the hydroxyl-to-carboxyl ratio becomes excessively high.

The DTG curves often indicate a two-step thermal degradation process, with major decomposition stages occurring at temperatures around 350° C. and 415° C. The DSC analysis of the post-cured ZCSX polyesters, as presented in FIG. 8B, typically reveals the absence of sharp melting peaks (Tₘ), which is characteristic of amorphous, highly crosslinked thermoset polymers. However, these polyesters exhibit well-defined glass transition temperatures (T_{g}), which are indicative of their elastomeric or glassy nature at different temperatures. The T_{g} values are dependent on the specific ZCSX composition. As seen in FIG. 8B, the T_{g} values may range, for example, from about 1.62° C. for ZCSX (1.5) to about 10.26° C. for ZCSX (0.5). A general trend observed is that an increase in zingerol content in the polyester formulation tends to shift the T_{g} to a lower temperature, suggesting that higher zingerol content may lead to increased flexibility in the polymer network due to factors like reduced crosslinking density or the plasticizing effect of zingerol moieties if not fully reacted at all hydroxyl sites.

While these specific characterization data are presented for zingerol-based ZCSX polyesters, it is anticipated that polyesters formed using other plant-derived diols from the group consisting of gingerols, zingerol, and reduced derivatives of paradol, zingerone, and shogaols, when incorporated into the Formula (I) architecture and synthesized via analogous melt polycondensation methods, would exhibit characteristic polyester spectroscopic features (e.g., ester carbonyls in FTIR, relevant proton signals in NMR reflecting the specific diol structure) and thermal properties that can be similarly evaluated.

### Properties and Performance of ZCSX Biodegradable Polyesters

The ZCSX biodegradable polyesters, synthesized as described herein, exhibit a remarkable range of physicochemical, mechanical, and biological properties that are highly tuneable by adjusting the molar ratios of the constituent monomers, particularly the zingerol. These properties underscore their significant potential for various biomedical applications.

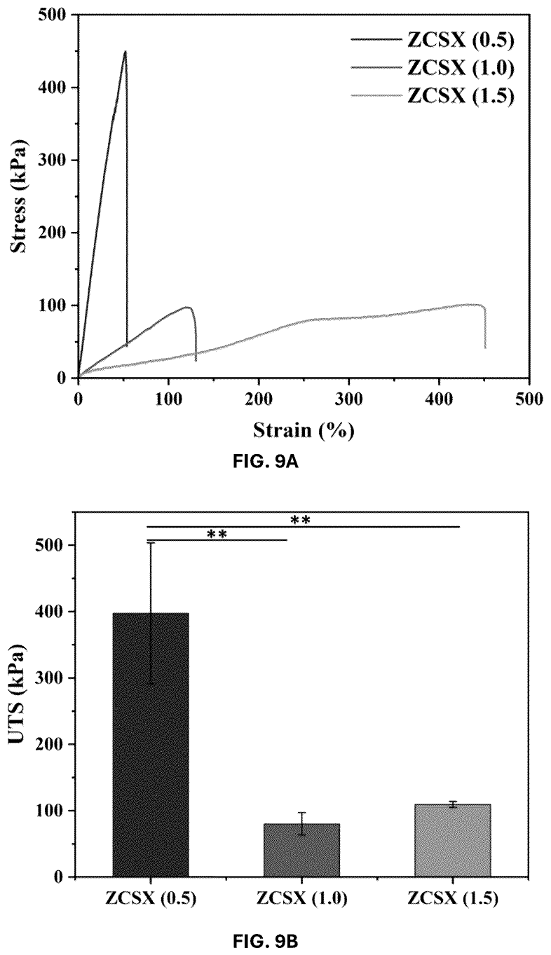

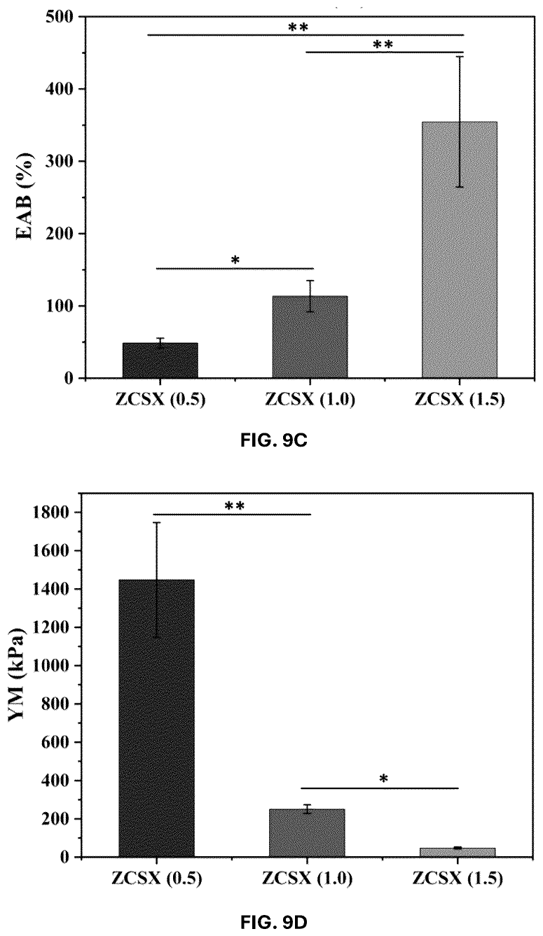

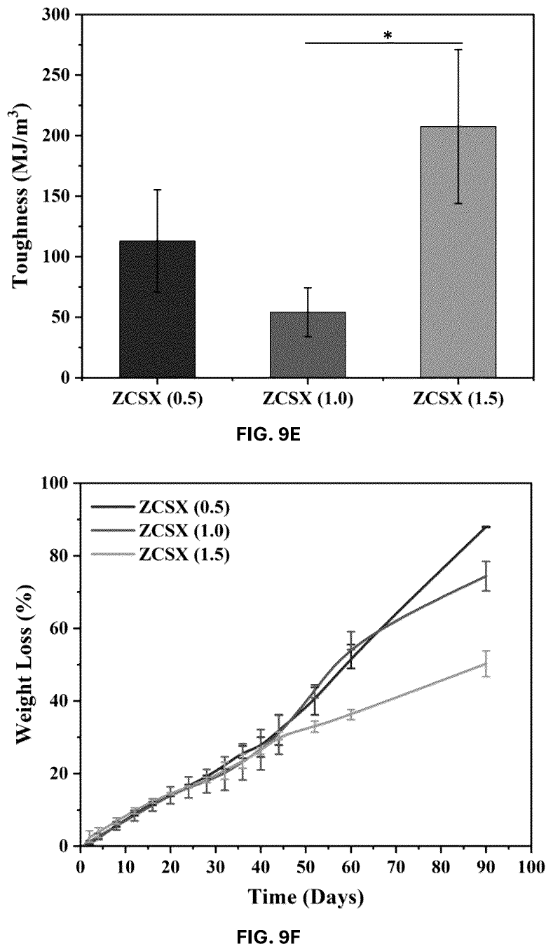

In certain embodiments, the mechanical properties of the ZCSX polyesters are thoroughly investigated to assess their suitability for applications such as soft tissue engineering. As shown in FIG. 9A, tensile testing of various ZCSX compositions, such as ZCSX (0.5), ZCSX (1.0) and ZCSX (1.5), where the number indicates the molar ratio of zingerol, reveals distinct stress-strain behaviours. These curves demonstrate that the mechanical response, including strength, stiffness, and elasticity, can be effectively modulated. For instance, the Ultimate Tensile Strength (UTS) of these polyesters, as comparatively shown in the bar graph of FIG. 9D, may range from approximately 79.99±16.86 kPa for ZCSX (1.0) to as high as 397.22±106.4 kPa for ZCSX (0.5), with ZCSX (1.5) showing an intermediate value of about 109.27±4.58 kPa. The higher UTS observed in compositions like ZCSX (0.5), which has a lower zingerol content relative to the crosslinking agents, is generally attributed to a higher degree of crosslinking within the polymer network.

The elasticity and stretchability of these ZCSX polyesters are further characterized by their Elongation at Break (EAB), presented in FIG. 9B. Different embodiments show a wide range of EAB values; for example, ZCSX (0.5) may exhibit a lower EAB of around 48.68±6.79%, which is clearly indicative of a more rigid material, whereas ZCSX (1.5), with a higher zingerol content, can demonstrate an excellent stretchability with an EAB as high as 354.48±90.18%. ZCSX (1.0) may show an intermediate EAB of about 113.47±21.66%. This enhanced elasticity in compositions with higher zingerol ratios may be due to a combination of factors, including potentially lower crosslink density and the presence of more unreacted hydroxyl groups from zingerol that can form a flexible hydrogen-bonding network, acting as sacrificial bonds.

The stiffness of these materials, represented by Young's Modulus (YM), also varies significantly with composition, as shown in FIG. 9C. For instance, ZCSX (0.5) can demonstrate the highest Young's modulus (e.g., 1447.72±299.86 kPa), while ZCSX (1.0) and ZCSX (1.5) may exhibit progressively lower moduli (e.g., 250.45±22.89 kPa and 47.68±5.34 kPa, respectively).

The toughness of these polyesters, which signifies their ability to absorb energy before fracture, is illustrated in FIG. 9E. In some embodiments, highly stretchable compositions like ZCSX (1.5) can exhibit the highest toughness (e.g., 207.49±63.68 MJ/m³), while ZCSX (0.5) and ZCSX (1.0) may possess toughness values of about 112.96±42.29 MJ/m³ and 54.05±20.18 MJ/m³, respectively. The range of these mechanical properties indicates that ZCSX polyesters can be tailored to mimic various soft tissues in the human body, including but not limited to soft collagenous bone, components of the cervical spine, articular cartilage, ligaments, aorta, liver, bladder tissue, breast tissue, temporal cartilage, and septal muscle, thereby highlighting their potential in diverse tissue engineering applications.

It is further contemplated that variations in the specific plant-derived diol structure within Formula (I) would allow for the fine-tuning of these mechanical properties while retaining the overall beneficial material characteristics demonstrated by the zingerol-based ZCSX examples.

The biodegradability of the ZCSX polyesters is a crucial feature for their intended use as temporary medical implants or scaffolds. In-vitro hydrolytic degradation studies, typically performed in Dulbecco's phosphate-buffered saline (DPBS, pH 7.4) at 37° C., demonstrate that these materials undergo gradual degradation over time, as shown by the weight loss profiles in FIG. 9F. The degradation mechanism primarily involves the hydrolysis of ester bonds within the polyester network.

In many embodiments, an initial phase of relatively slow degradation is observed (e.g., approximately 20% weight loss across all tested ZCSX compositions after about one month), which can be attributed to their highly crosslinked structure that may initially limit the rapid penetration of water molecules into the polymeric matrix. However, as hydrolysis progresses, a more notable increase in degradation becomes apparent. For example, after about 40 days, and continuing up to 90 days, ZCSX (0.5) and ZCSX (1.0) compositions might degrade by as much as 90% and 75%, respectively. In comparison, ZCSX (1.5) composition might degrade by approximately 50% within the same 90-day period.

This trend in degradation rates can be explained by the interplay between the hydroxyl-to-carboxylic acid group ratio, the resulting crosslinking density, and the overall hydrophilicity of the polymer. For instance, in a ZCSX (1.5) composition where the hydroxyl-to-carboxylic acid ratio may be greater than 1, implying more free hydroxy groups than ester linkages initially available for hydrolysis, there might be a faster initial surface degradation. However, as these accessible alcohol groups are lost, the material may become relatively more hydrophobic due to the higher proportion of aromatic zingerol groups compared to more hydrophilic compositions, potentially leading to slower degradation in the long run.

Conversely, ZCSX (0.5) and ZCSX (1.0) compositions, with hydroxyl-to-acid group ratios of less than or equal to 1, respectively, might be more hydrophilic overall, facilitating more consistent water penetration and hydrolysis over time. This tuneable degradation profile allows for the material to be engineered to match the healing rate of the target tissue.

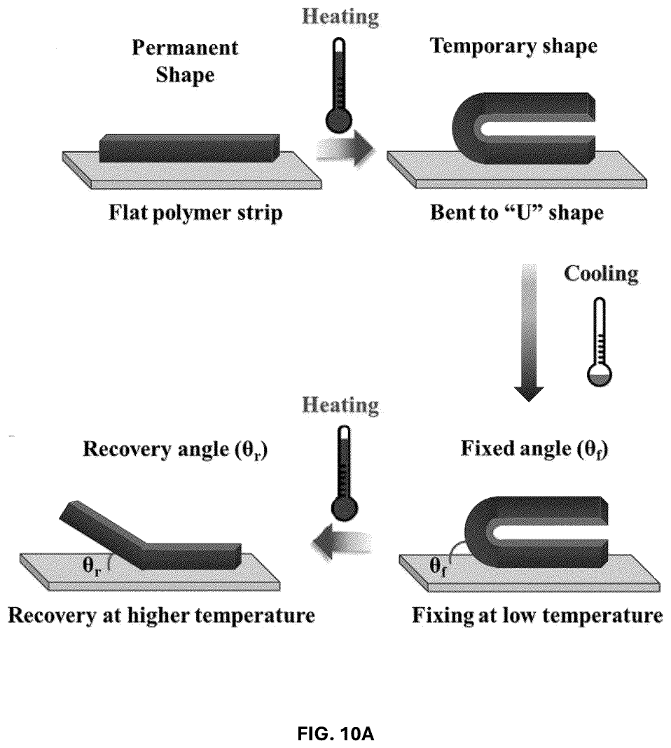

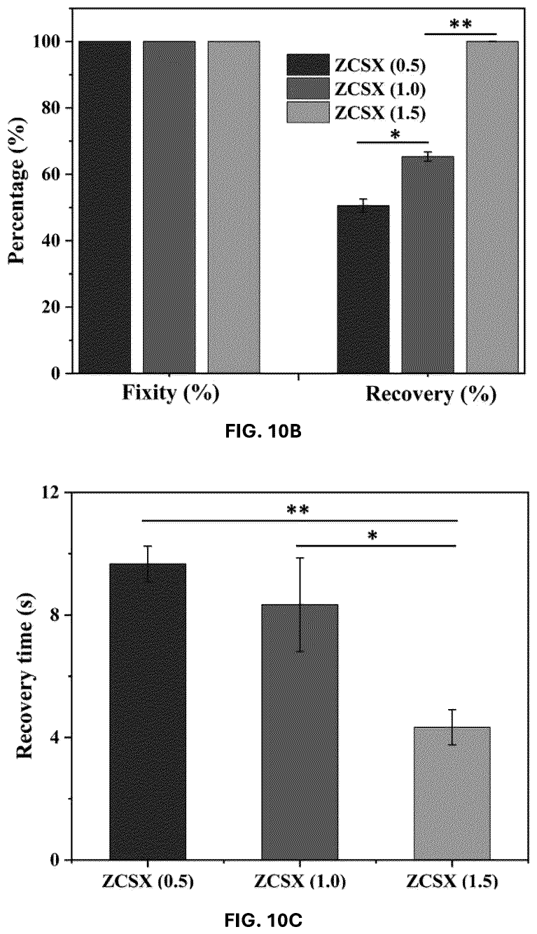

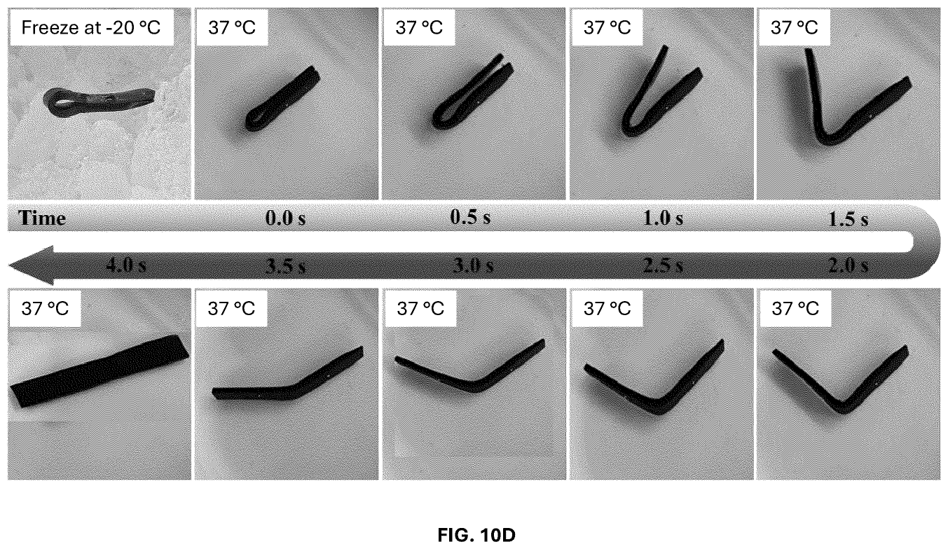

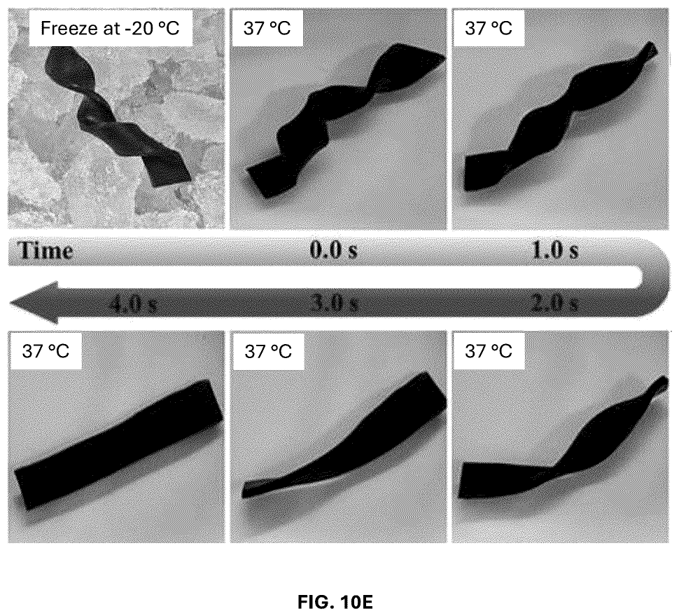

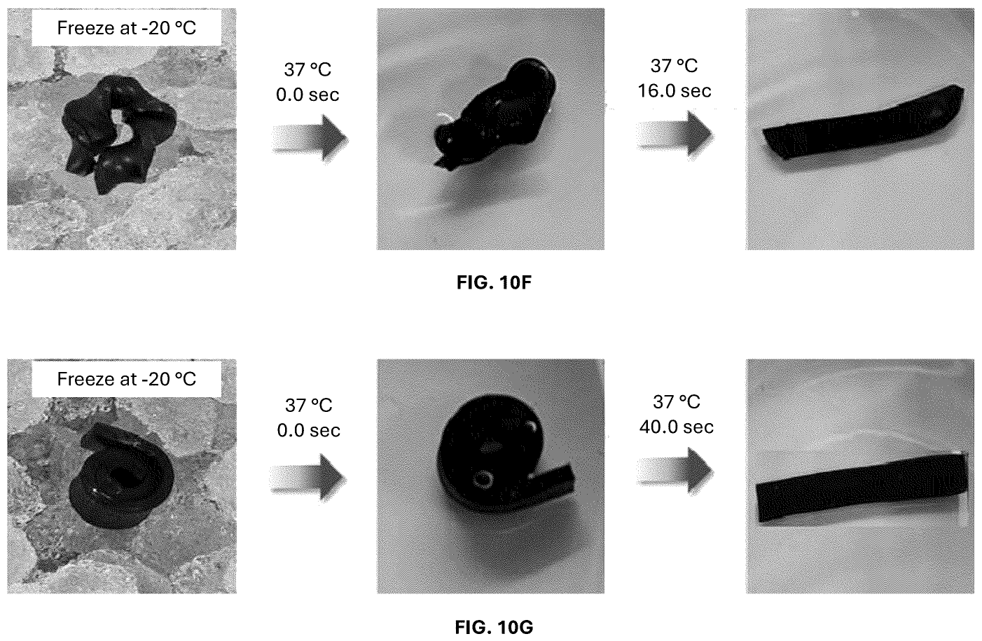

An advantageous property of certain embodiments of the ZCSX polyesters is their excellent shape memory behaviour, which is highly beneficial for applications in minimally invasive surgery and for deployable medical devices. This property is typically thermoresponsive, allowing the material to be programmed into a temporary, often compact, shape and then recover its original, permanent shape upon exposure to a stimulus such as body temperature (˜37° C.). The assessment of these shape memory properties often involves U-bending tests, where parameters such as the fixed angle (θ_{f}) in the temporary deformed state and the recovery angle (θᵣ) after stimulation are measured, as schematically illustrated in FIG. 10A.

Embodiments of the ZCSX polyesters can demonstrate very high shape fixity ratios, typically ranging from about 98% to 100%, indicating their ability to effectively maintain the temporary deformed shape. Furthermore, they can exhibit high shape recovery percentages, often reaching 100% upon triggering, as demonstrated by the data in FIG. 10C, which shows fixity and recovery ratios after approximately 4 seconds of recovery time.

The time required to achieve full (100%) shape recovery is also an important parameter and can be quite rapid for these materials, as shown in FIG. 10B. For example, a ZCSX (1.5) composition may fully recover its original shape in as little as approximately 4 seconds, whereas ZCSX (1.0) and ZCSX (0.5) compositions might take about 8 seconds and 10 seconds, respectively. This trend suggests that increased zingerol content, which can lead to increased flexibility and potentially a lower effective transition temperature or faster chain mobility, facilitates quicker recovery.

The visual manifestation of this shape memory effect is powerfully demonstrated through series of digital photographic images. FIG. 10D sequentially documents the recovery of a ZCSX (1.5) polyester strip from a U-bent temporary shape back to its original flat shape when immersed in 37° C. water after being fixed at −20° C. Moreover, the versatility of this shape memory effect is shown in FIGS. 10E-10G, which illustrate the ability of ZCSX polyesters to recover their original programmed shapes from more complex temporary configurations such as a helical form (FIG. 10E), a U-shaped helix (FIG. 10F), and a tightly wound coil (FIG. 10G), with recovery times varying depending on the complexity of the deformed shape, e.g., ˜4 sec. for helical, ˜16 sec. for U-shaped helix, ˜40 sec. for coil for ZCSX (1.5).

The adhesive nature of some compositions like ZCSX (1.5) might also influence the recovery from very compact shapes. The underlying mechanism for these excellent shape memory properties in ZCSX polyesters is attributed to their specific network architecture, which combines rigid aromatic hard segments derived from the zingerol moieties (acting as netpoints that define the permanent shape) with flexible aliphatic soft segments (e.g., from sebacic acid, allowing for deformation into temporary shapes), all interconnected by chemical crosslinks formed by citric acid and xylitol, which stabilize the permanent shape.

The ZCSX polyesters of the present invention also exhibit a suite of highly favourable biological properties, rendering them particularly suitable for biomedical applications. Indeed, a significant feature, in many embodiments, is their inherent antibacterial activity. As demonstrated through various microbiological assays, these polyesters show good efficacy against both Gram-positive bacteria, such as Bacillus subtilis, and Gram-negative bacteria, such as Escherichia coli.

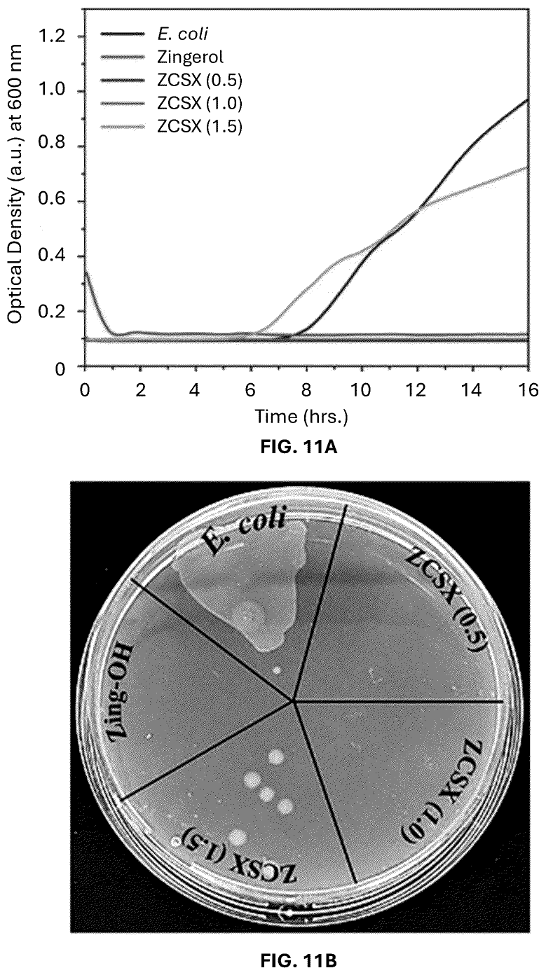

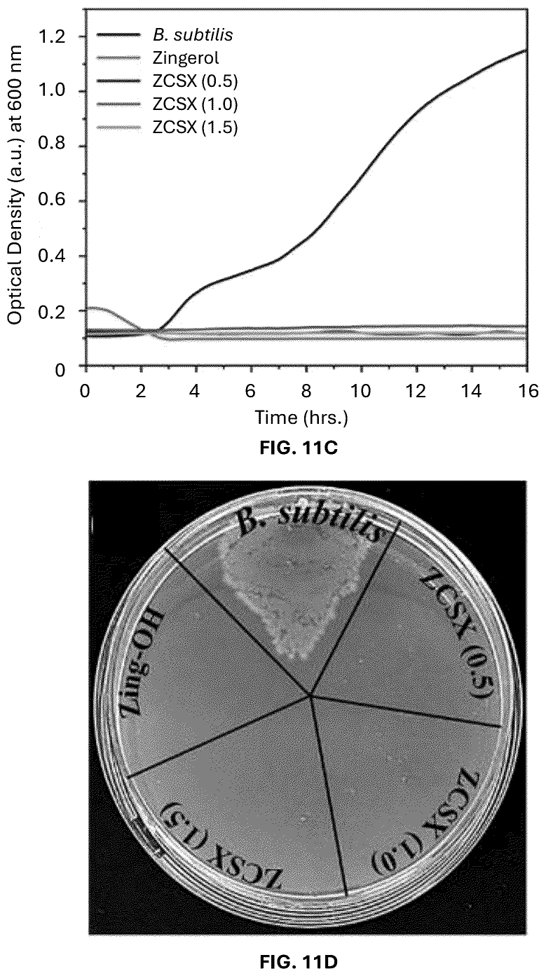

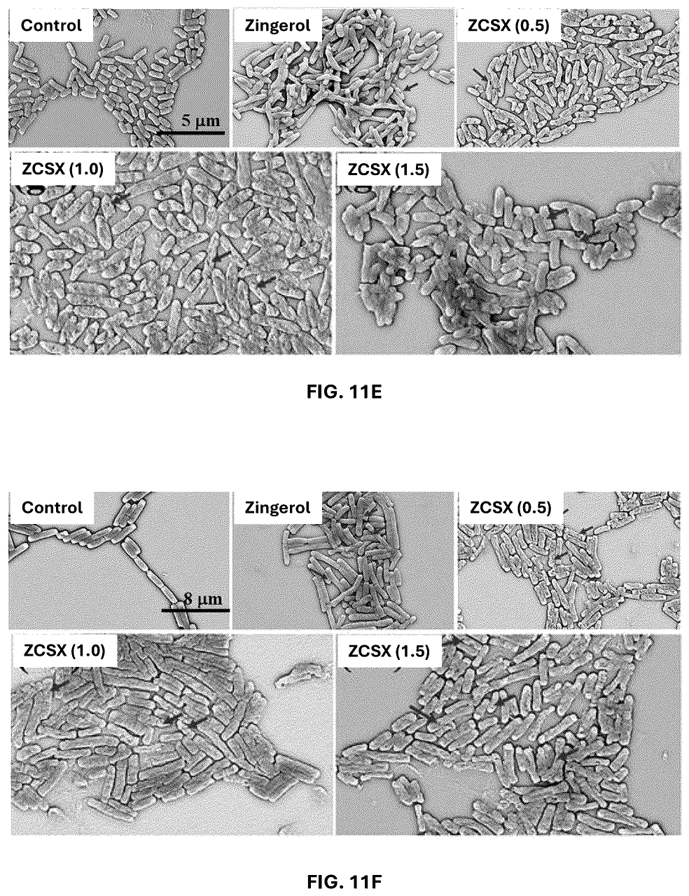

The effect on bacterial proliferation is shown in bacterial growth curve analyses presented in FIG. 11A (for E. coli) and FIG. 11C (for B. subtilis), where the presence of ZCSX polyesters leads to a significant inhibition of bacterial growth compared to control conditions. This antibacterial effect is further visually confirmed by colony formation assays, depicted in FIG. 11B (for E. coli) and FIG. 11D (for B. subtilis), which show a marked reduction in the number of viable bacterial colonies formed on agar plates after the bacteria have been in direct contact with the polyester surfaces. Some ZCSX compositions, particularly those other than ZCSX (1.5) in certain tests, may even lead to complete eradication of bacteria upon direct contact.

The bactericidal nature of these ZCSX polyesters, complementing the growth curve analyses (FIGS. 11A and 11B) and colony formation assays (FIGS. 11C and 11D), was further corroborated by live/dead staining assays using fluorescence microscopy. These microscopic evaluations for both E. coli and B. subtilis after exposure to the polyesters consistently revealed a predominant population of dead or membrane-compromised bacteria (e.g., exhibiting red or yellow fluorescence with typical live/dead stain kits) compared to control bacterial populations which showed primarily live bacteria (e.g., exhibiting green fluorescence). These observations provide additional visual confirmation of the antibacterial efficacy of the ZCSX polyesters.

To elucidate the mechanism of this antibacterial action, Scanning Electron Microscopy (SEM) has been employed to examine the morphology of bacteria after interaction with the ZCSX polyesters. As shown in FIG. 11E for E. coli and FIG. 11F for B. subtilis, the SEM images often reveal significant damage to the bacterial cell membranes, including the formation of pores or complete lysis, suggesting that the antibacterial effect involves disruption of bacterial cell integrity. This membrane-disruptive activity is likely attributable to the zingerol component within the polyester structure, as zingerol and related ginger compounds are known to interact with and compromise bacterial membranes.

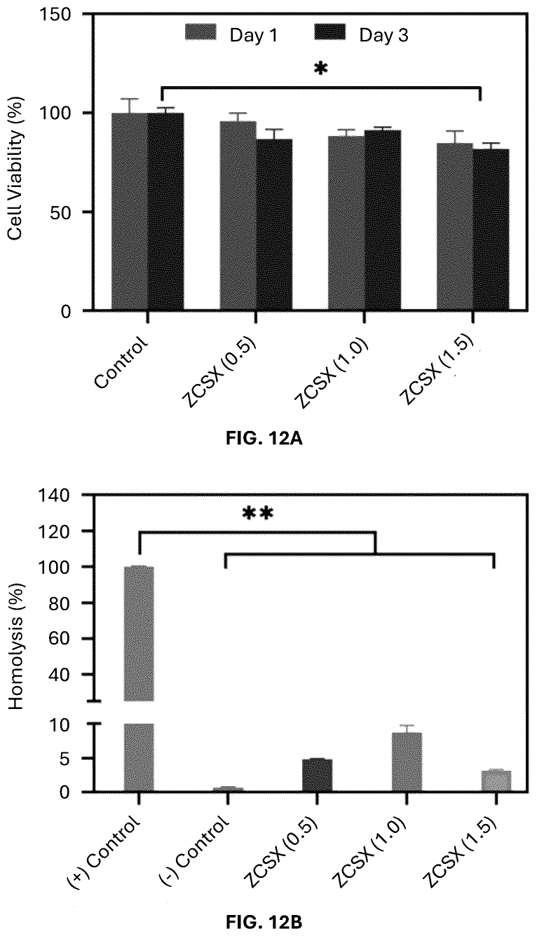

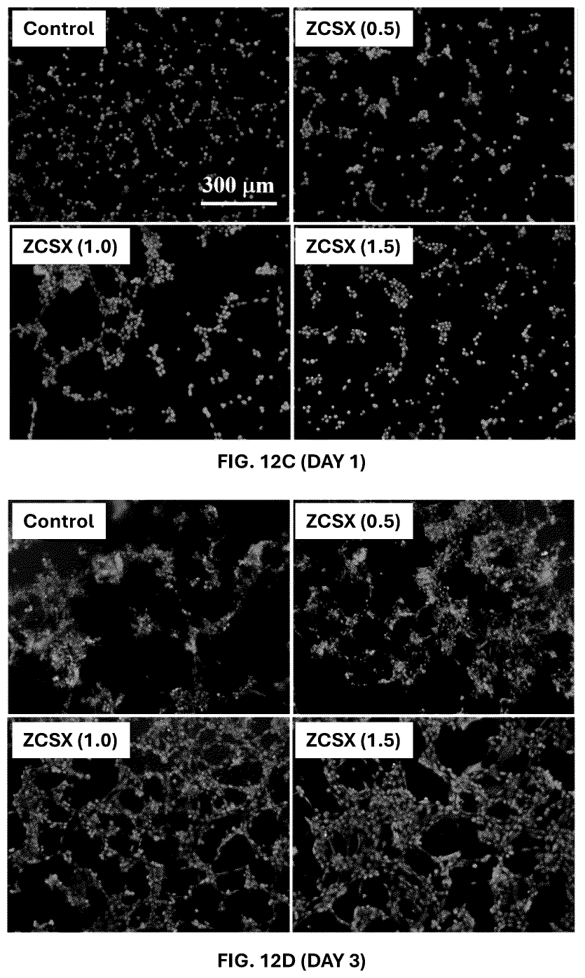

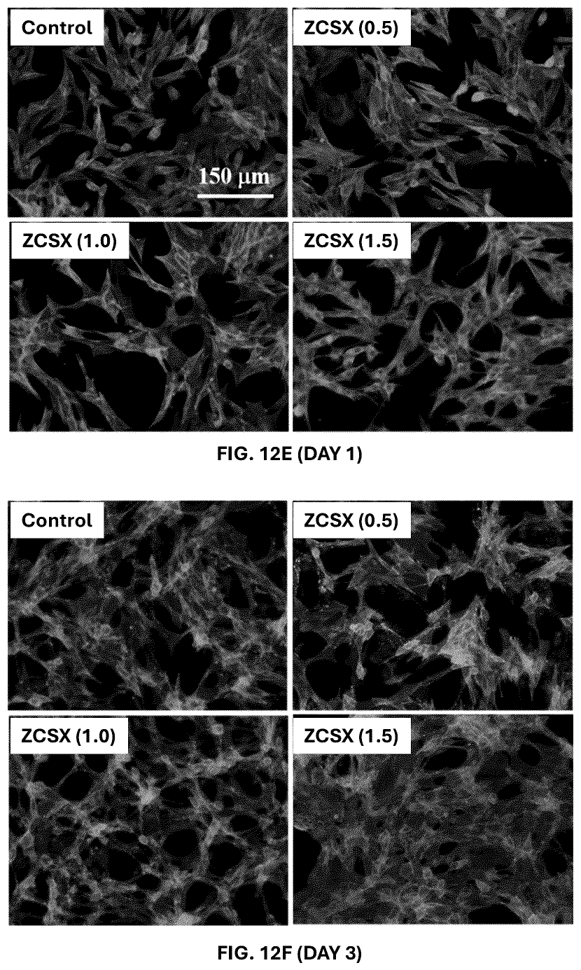

In addition to antibacterial properties, the cytocompatibility and hemocompatibility of the ZCSX polyesters are critical for their safe use in biomedical applications. In various embodiments, these polyesters demonstrate good cytocompatibility when evaluated with mammalian cell lines, such as NIH/3T3 murine fibroblasts. As shown by Alamar Blue cell viability assays in FIG. 12A, conditioned media prepared by incubating the ZCSX polyesters in cell culture medium generally supports robust cell viability and proliferation over several days (e.g., 1 and 3 days), with viability percentages often comparable to or even exceeding those of control cells cultured in fresh medium alone. For instance, ZCSX (0.5) shows around 95% viability, while ZCSX (1.0) and ZCSX (1.5) show around 90% and 85% viability respectively, relative to control, indicating that any leachable or degradation products from these polyesters are not significantly cytotoxic.

This is further corroborated by live/dead cell staining assays, such as those depicted in FIG. 12C (1-day exposure) and FIG. 12D (3-day exposure), where cells exposed to conditioned media from ZCSX polyesters predominantly stain as viable (green fluorescence) with minimal dead cells (red fluorescence), similar to control cell populations. Furthermore, cell morphology studies, involving staining for cellular components like F-actin filaments (e.g., with Alexa Fluor 546-coupled phalloidin) and nuclei (e.g., with DAPI), show that cells cultured in the presence of ZCSX polyester-conditioned media maintain a healthy, well-spread morphology and exhibit normal intercellular connections and filopodial protrusions, as illustrated in FIG. 12E (1-day) and FIG. 12F (3-day). This indicates that the materials do not adversely affect cell structure or behaviour.

Regarding hemocompatibility, which is crucial for materials that may come into contact with blood, the ZCSX polyesters generally perform very well. As clearly seen in FIG. 12B, haemolysis assays conducted by incubating the polyesters with fresh human red blood cells typically result in low percentages of haemolysis. For example, different ZCSX compositions may exhibit haemolysis rates ranging from about 3.12% for ZCSX (1.5) to about 4.81% for ZCSX (0.5), and about 8.75% for ZCSX (1.0). While some compositions slightly exceed the ideal <5% threshold, others like ZCSX (1.5) are well within it, indicating substantial hemocompatibility, especially when compared to positive controls, for example, deionized water causing complete lysis, and negative controls, for example phosphate-buffered saline causing minimal lysis. This suggests that these polyesters pose a low risk of causing adverse haematological reactions when used in vivo.

Furthermore, certain embodiments of the ZCSX polyesters of the invention have demonstrated a promising potential to promote wound healing. This property is often evaluated using in-vitro scratch wound healing assays with fibroblast cell lines like NIH/3T3. In such assays, a “scratch” or cell-free area is created in a confluent cell monolayer, and the ability of cells to migrate and close this gap is monitored over time.

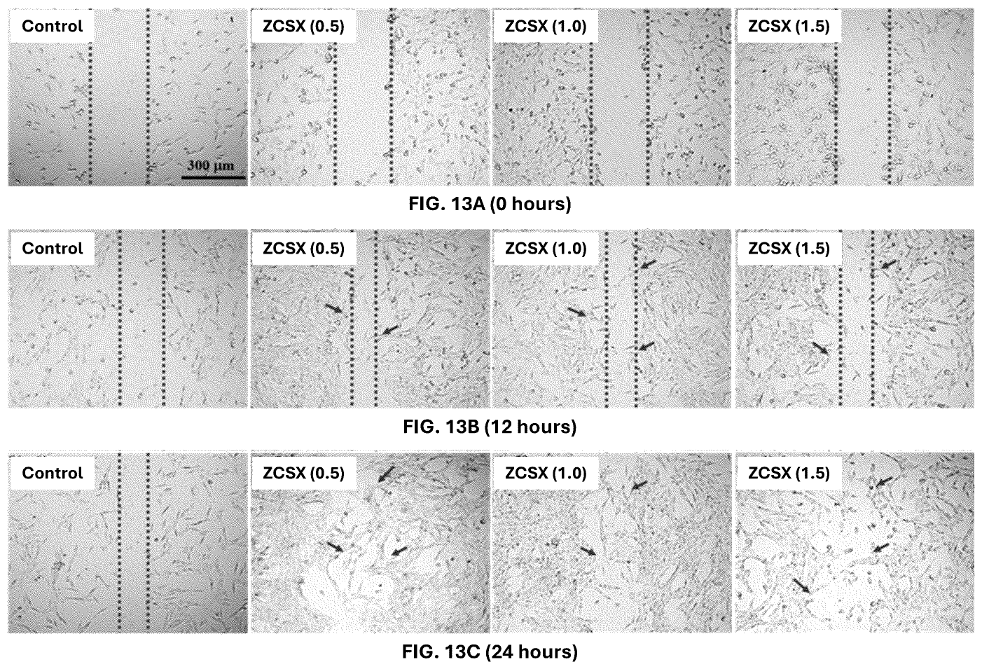

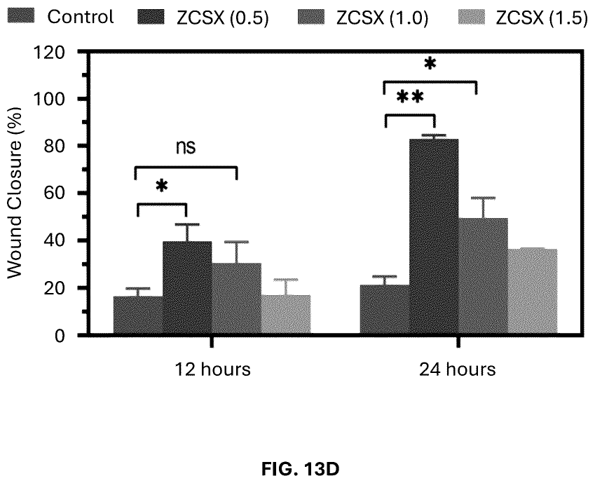

As illustrated by the representative microscopy images in FIG. 13A (0 hours), FIG. 13B (12 hours), and FIG. 13C (24 hours), cells treated with conditioned media obtained from ZCSX polyesters often exhibit enhanced migration into the scratch area and faster wound closure compared to cells treated with control media.

The quantitative analysis of this effect, presented as percentage wound closure in FIG. 13D, can show significantly accelerated healing rates. For instance, conditioned media from ZCSX (0.5) might lead to approximately 39.71±7.10% wound closure within 12 hours and about 82.98±1.61% closure within 24 hours. In contrast, control cells might only achieve about 16.56±3.91% closure in 12 hours and 21.33±3.52% in 24 hours. Other compositions like ZCSX (1.0) and ZCSX (1.5) also show enhanced wound closure compared to control, for example, ZCSX (1.0) achieving about 30.44% (12 h) and 49.62% (24 h) closure, and ZCSX (1.5) achieving about 17.16% (12 h) and 36.58% (24 h) closure. These findings suggest that the ZCSX polyesters or their degradation products may release factors or possess surface characteristics that promote cell migration and can aid in the wound healing process.

It is contemplated by the inventors that by substituting zingerol with other structurally related plant-derived diols from the group consisting of gingerols, zingerol, and reduced derivatives of paradol, zingerone, and shogaols, in the synthesis of polyesters according to Formula (I), one could modulate these properties while retaining the overall beneficial characteristics.

For example, variations in the side chains or flexibility of these alternative diols might influence the resulting polymer's T_{g}, mechanical strength, elasticity, degradation kinetics, and even the specific bioactivity profile (e.g., antioxidant or antibacterial potency related to the specific phenolic structure). Such modulations are within the scope of the present invention and would allow for further tailoring of the polyesters for specific biomedical applications. The general synthetic approach and the fundamental polyester structure would remain, providing a platform for generating a family of related biomaterials.

### Applications of ZCSX Biodegradable Polyesters

The unique amalgamation of properties exhibited by the ZCSX biodegradable polyesters of the present invention, particularly those derived from zingerol and related plant-derived diols as described herein, including their derivation from renewable resources like zingerol, their synthesis via an environmentally friendly catalyst-free and solvent-free melt polycondensation process, their tuneable mechanical characteristics that can mimic various biological tissues, their excellent shape memory behaviour, inherent antibacterial activity, good cytocompatibility and hemocompatibility, and potential to promote wound healing, renders them exceptionally suitable for a broad spectrum of applications, particularly within the biomedical and tissue engineering domains.

In various embodiments, these innovative ZCSX polyesters can be effectively utilized for creating tissue engineering scaffolds. Their tuneable mechanical properties allow for the design of scaffolds that can match the requirements of diverse human tissues targeted for regeneration. This includes applications for soft tissues such as liver, uterus, bladder, and breast tissue, as well as for more structurally demanding tissues like temporal cartilage, nasal cartilage, soft collagenous bone, components of the cervical spine, articular cartilage, ligaments, and even vascular tissues like the aorta. The biodegradability of these scaffolds ensures that they can provide temporary support for cell growth and tissue formation, eventually being resorbed by the body as the new tissue matures.

Furthermore, the pronounced shape memory properties of these polyesters make them ideal candidates for manufacturing shape memory items intended for biomedical use. This is particularly advantageous for developing devices that can be employed in minimally invasive surgical (MIS) procedures. Such devices can be fabricated in a permanent functional shape, then temporarily deformed into a compact or pre-programmed secondary shape for easier insertion through small incisions or catheters, and subsequently triggered (e.g., by body temperature) to recover their original functional shape in situ, facilitating complex procedures and potentially reducing patient trauma and recovery time.

The inherent antibacterial activity demonstrated by the ZCSX polyesters is another significant advantage, allowing for their use in manufacturing materials and devices where prevention of bacterial infection is critical. This includes applications such as coatings for medical implants, components of wound dressings, or as bulk materials for implants themselves, thereby reducing the risk of device-associated infections, which are a major cause of implant failure and patient morbidity.

Given their excellent biocompatibility, biodegradability, and positive interactions with cells, including promoting cell migration, these polyesters are also well-suited for various regenerative medicine and wound healing applications. They can be formulated into films, hydrogels (if appropriately modified), porous scaffolds, or other constructs designed to accelerate tissue repair, protect wound sites, and facilitate the organized regeneration of damaged tissues.

The overall profile of these materials also makes them suitable for fabricating a wide range of implantable medical items where biodegradability, tailored mechanical performance, and biological safety are paramount considerations. The fact that these advanced polyesters can be synthesized using a solvent-free and catalyst-free process further enhances their appeal for biomedical applications. This environmentally friendly “green chemistry” approach not only reduces the manufacturing footprint but also minimizes the risk of residual toxic catalysts or organic solvents leaching from the final biomedical product, potentially simplifying purification steps and improving the overall safety and biocompatibility profile of the implanted material.

### 3D-Printable Zingerol-Derivative Compositions and Objects Therefrom

In another aspect, the present invention provides novel photopolymerizable compositions derived from zingerol, specifically designed for additive manufacturing techniques such as 3D printing. These compositions, upon polymerization, yield three-dimensional objects with tuneable properties suitable for various applications, particularly in the biomedical field. This aspect of the invention encompasses the novel zingerol derivative monomers, the photopolymerizable resin compositions formulated therefrom, methods for their preparation, the 3D-printed objects themselves, methods of manufacturing said objects, and their advantageous properties and uses.

### Photopolymerizable Zingerol Derivative Monomers or Crosslinkers

A key feature of this aspect of the invention is the development of novel zingerol derivative monomers or crosslinkers. These monomers or crosslinkers are synthesized by chemically modifying zingerol (Zing-OH), as described above, to incorporate photopolymerizable (meth)acrylate moieties.

According to certain embodiments, these zingerol derivative monomers or crosslinkers having Formula (II) may be represented by the following structure:

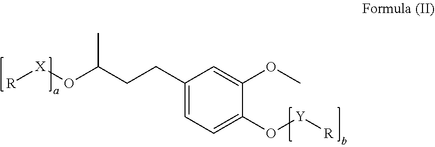

wherein the oxygen atom attached to X and the oxygen atom attached to Y are derived from the native secondary aliphatic hydroxyl group and the native phenolic hydroxyl group of zingerol, respectively;
a and b are independently 0 or 1, with the proviso that a+b is at least 1;
each R independently represents a photopolymerizable (meth)acrylate group of the formula

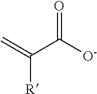

wherein R′ is H or methyl;
each X independently represents a divalent linking group such that the moiety —O—X—R forms a functionalized zingerol aliphatic hydroxyl group wherein an ether, ester, or urethane linkage connects said oxygen to said R group via said linking group X; and
each Y independently represents a divalent linking group such that the moiety —O—Y—R forms a functionalized zingerol phenolic hydroxyl group wherein an ether, ester, or urethane linkage connects said oxygen to said R group via said linking group Y.

Essentially, at least one of the native hydroxyl groups of zingerol (either the aliphatic secondary alcohol or the phenolic alcohol, or both) is functionalized. This functionalization connects a photopolymerizable (meth)acrylate group to the zingerol oxygen via a linking group, wherein the nature of this connection involves the formation of an ether, an ester, or a urethane linkage.

In preferred embodiments, these zingerol derivative monomers include specific structures, wherein:
(i) for an ester-type linkage derived from the reaction of zingerol with, X or Y is a direct bond, resulting in a structure:

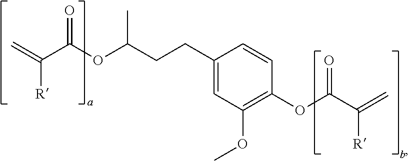

or
(ii) for an ether-type linkage derived from the reaction of zingerol with glycidyl (meth)acrylate, wherein the (meth)acrylate group is —C(O)—C(R′)═CH₂ and R′ is H or methyl, X or Y is —CH₂—CH(OH)—CH₂—O—; or
(iii) for a urethane-type linkage derived from the reaction of zingerol with an isocyanatoalkyl (meth)acrylate, X or Y is —C(═O)—NH—R″—O—, wherein R″ is a C₁-C₆ alkylene group.

In specific embodiments, these three options (i), (ii) and (iii) are exemplified by zingerol-methylacrylate (ZES), zingerol-glycidyl methacrylate (ZET), and zingerol-urethane (ZUR), respectively.

ZES type derivative (option i) is formed where a zingerol hydroxyl oxygen is linked via an ester bond directly to the carbonyl of a (meth)acrylate moiety. The structure is Zingerol —O—C(═O)—C(R′)═CH₂, where R′ is typically methyl.

ZET type derivative (option ii) is formed where a zingerol hydroxyl oxygen is linked, for example, via an ether bond resulting from reaction with the epoxide of glycidyl (meth)acrylate, to a 2-hydroxypropylene group, which in turn is typically linked via an ester bond (native to the methacrylate) to the (meth)acrylate moiety. The overall structure can be represented as Zingerol —O—CH₂—CH(OH)—CH₂—O—C(═O)—C(R′)═CH₂, where R′ is typically methyl. The primary new linkage formed at the zingerol oxygen when reacting with glycidyl methacrylate is an ether bond.

ZUR type derivative (option iii) is formed where a zingerol hydroxyl oxygen is linked via a urethane bond (—O—C(═O)—NH—) to an alkyl group (e.g., an ethylene group from isocyanatoethyl methacrylate, IEMA), which in turn is linked (often via an ester bond native to the IEMA) to a (meth)acrylate moiety. The structure can be represented as Zingerol —O—C(═O)—NH—R″—O—C(═O)—C(R′)═CH₂, where R″ is a divalent organic radical like an ethylene group, and R′ is typically methyl.

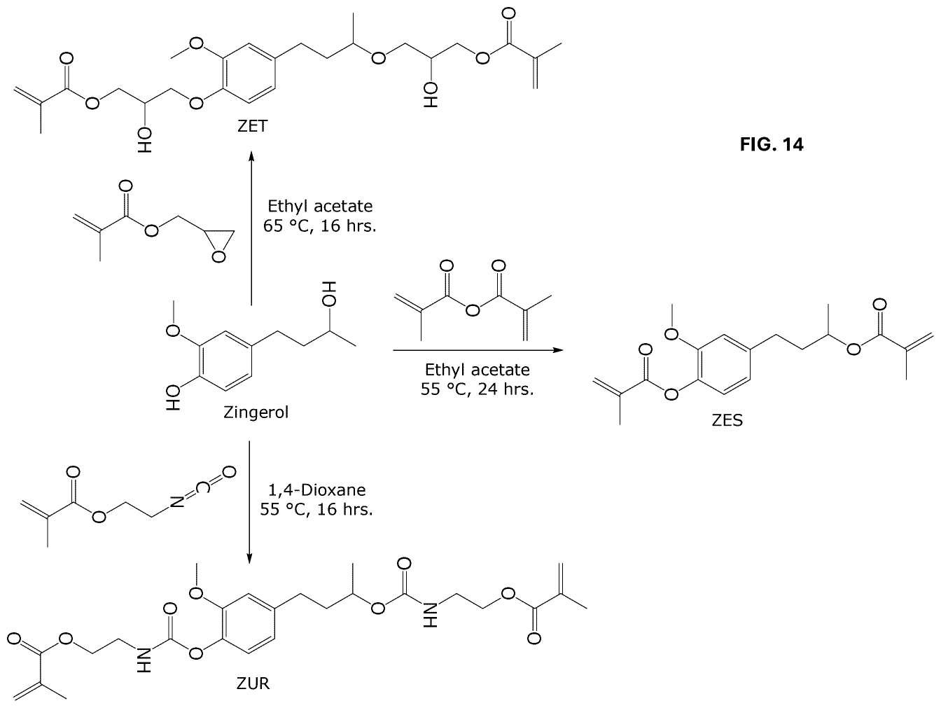

The chemical synthesis schemes for preparing ZET, ZES, and ZUR derivatives from zingerol are illustrated in FIG. 14. The subsequent photopolymerization of these (meth)acrylate-functionalized zingerol derivatives to form crosslinked polymer networks is schematically shown in FIG. 15.

### Preparing Photopolymerizable Zingerol Derivative Monomers or Crosslinkers

According to another aspect of the invention, a method for preparing these photopolymerizable zingerol derivative monomers or crosslinkers is provided. This method generally involves reacting zingerol with a suitable reagent capable of introducing the photopolymerizable (meth)acrylate group and forming the desired ether, ester, or urethane linkage at one or both of the zingerol's hydroxyl sites.

In specific embodiments, for preparing ZET-type derivatives (ether linkage formation at the zingerol oxygen, leading to a (meth)acrylate end-group), zingerol (for example, 1 equivalent) is reacted with glycidyl (meth)acrylate (for example, about 2.0 to 3.0 equivalents, typically about 2.5 equivalents, if aiming for difunctionalization). This reaction may be carried out in the presence of a base catalyst such as triethylamine (TEA, for example, about 0.1 equivalents) and in a suitable solvent like ethyl acetate (EA, for example, about 3 volumes relative to zingerol). The reaction is typically conducted under an inert atmosphere (for example, N₂) at an elevated temperature (for example, about 50-70° C., such as 60° C.) with stirring for a sufficient period (for example, about 12-24 hours, such as 18 hours), often under reflux. Workup involves procedures like extraction (for example, EA/brine) and solvent evaporation, followed by washing (for example, with hexane to remove unreacted glycidyl (meth)acrylate) to yield the ZET resin.

For preparing ZES-type derivatives (ester linkage formation), zingerol (for example, 1 equivalent) is reacted with a (meth)acrylating agent such as (meth)acrylic anhydride (for example, about 2.5 to 3.5 equivalents, typically about 3 equivalents). This esterification is often facilitated by a catalyst, such as 4-dimethylaminopyridine (DMAP, for example, about 0.2 equivalents), and performed in a solvent such as ethyl acetate (EA, for example, about 4 volumes). The reaction is typically conducted at an elevated temperature (for example, about 50-70° C., such as 60° C.) with stirring for several hours (for example, about 12-20 hours, such as 16 hours). Workup usually involves extraction (for example, EA/H₂O) followed by purification, for instance, using flash chromatography (for example, with a hexane/EA solvent system like 7:3) to remove impurities and isolate the ZES resin.

For preparing ZUR-type derivatives (urethane linkage formation), zingerol (for example, 1 equivalent) is reacted with an isocyanatoalkyl (meth)acrylate, such as iso-cyanatoethyl methacrylate (IEMA, for example, about 2.0 to 2.5 equivalents, typically about 2.1 equivalents). This urethanisation reaction is often catalysed, for example, by stannous octoate (Sn(Oct)₂, for example, about 0.05 equivalents). A suitable solvent such as dry 1,4-dioxane (for example, about 5 volumes) may be used, and the reaction is typically carried out under an inert atmosphere (e.g., N₂) at a moderately elevated temperature (for example, about 50-60° C., such as 55° C.) with stirring for an extended period (for example, about 18-30 hours, such as 24 hours). After solvent evaporation, the resulting ZUR resin is typically washed (for example, with petroleum ether and hexane) to remove catalyst and unreacted IEMA.

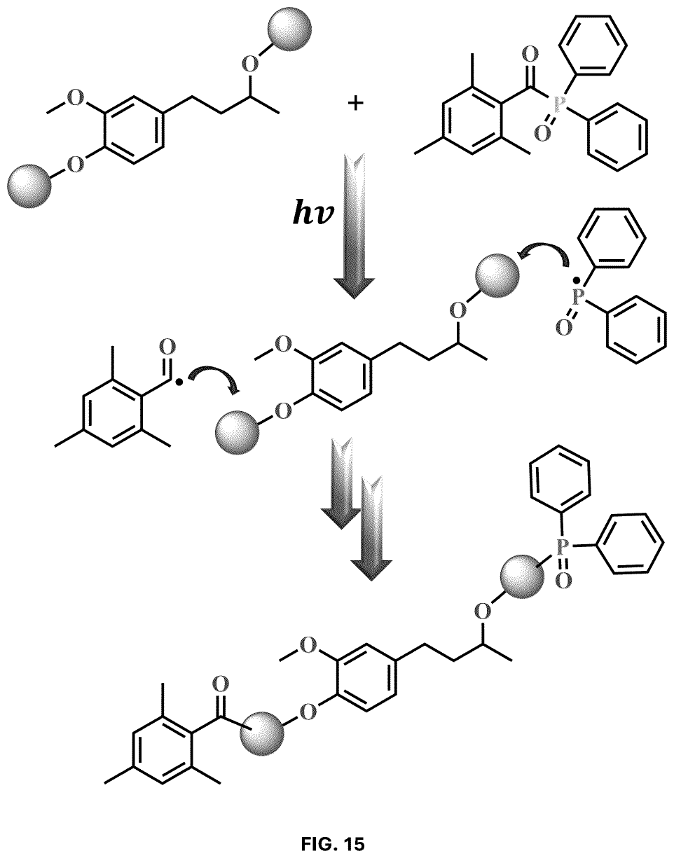

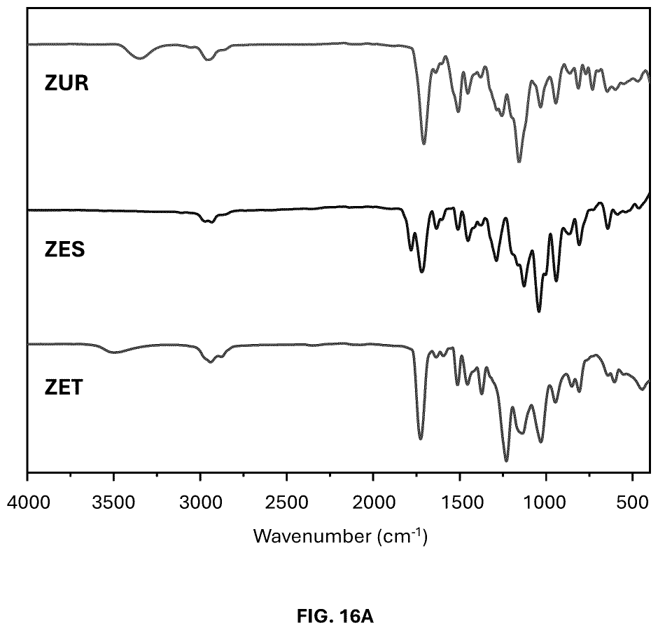

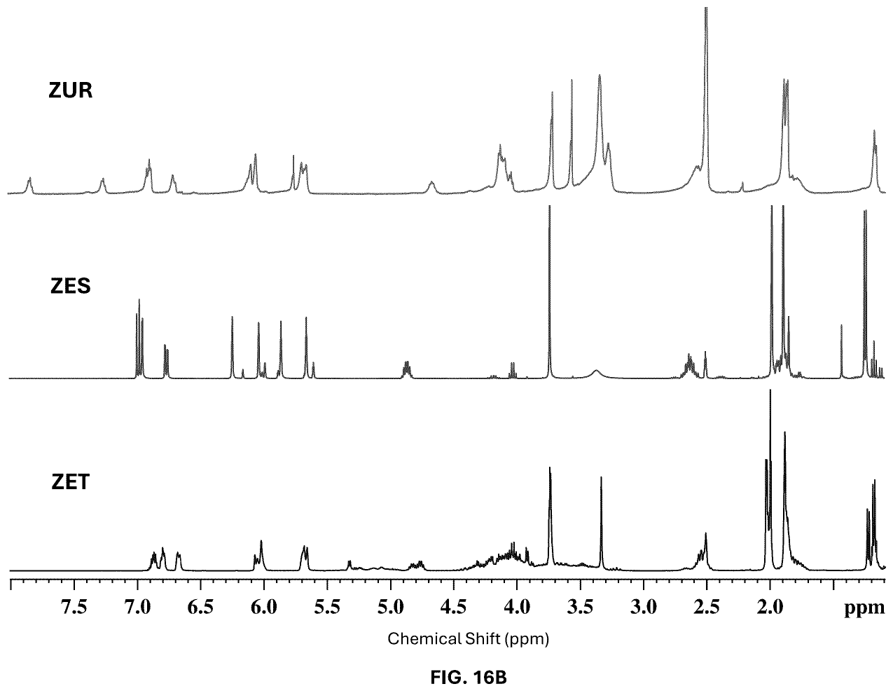

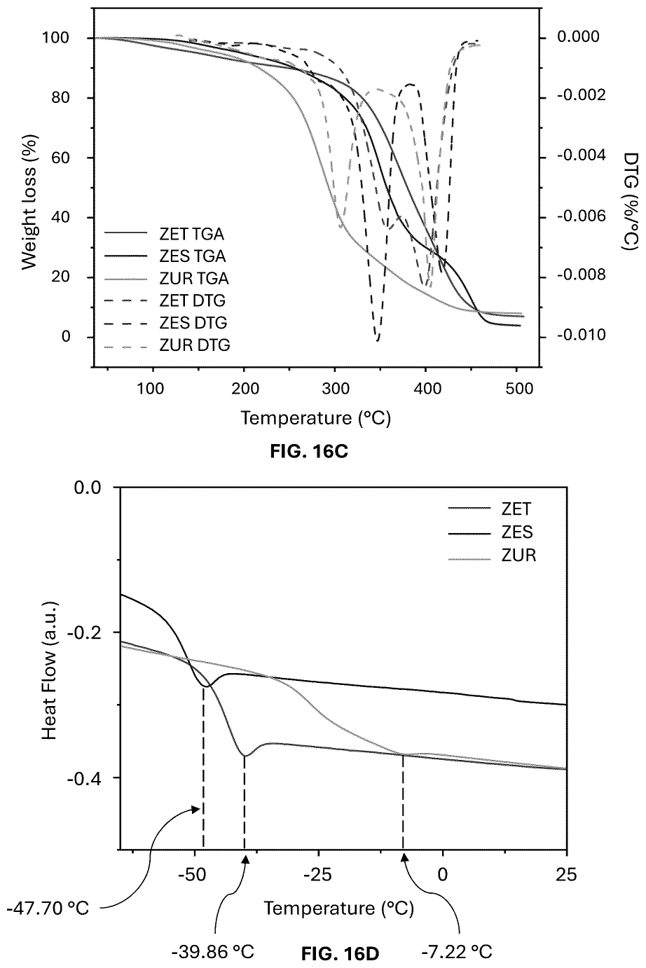

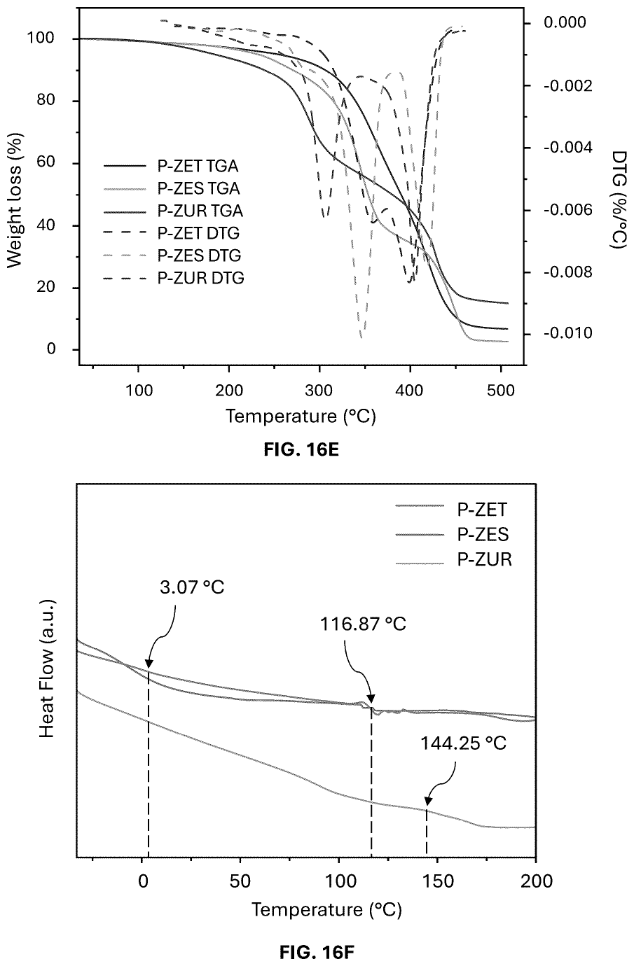

The successful synthesis and structure of ZET, ZES and ZUR are confirmed by various analytical techniques. FIG. 16A shows the representative FTIR spectra highlighting characteristic peaks such as carbonyl stretching (around 1705 cm⁻¹) from the (meth)acrylate and linker groups, acrylate (C═C) peaks (around 1650 cm⁻¹), and aromatic C—H bending (around 750 cm⁻¹) from the zingerol moiety. In addition, for the ZUR derivative, N—H stretching of the urethane bond (around 3300-3500 cm⁻¹) is also observed.

FIG. 16B presents 1H NMR spectra, where key signals include vinylic protons of the (meth)acrylate group (around 5.7-6.2 ppm), methyl protons of the methacrylate group (around 2 ppm), protons from the zingerol aromatic ring (around 6.8 ppm), and protons specific to the linking groups and any remaining hydroxyls. For example, in ZUR, —NH protons from the urethane bond may appear around 7.0-7.8 ppm.

The thermal properties of these unpolymerized resin compositions (ZET, ZES, ZUR) can be assessed by TGA/DSC, as shown in FIGS. 16C and 16D. For instance, the ZET, ZES, and ZUR resins may be stable up to about 300° C., 250° C., and 180° C., respectively, before significant degradation. DSC analysis can determine their melting points (ZET around −39.86° C., ZES around −47.70° C., ZUR around −7.22° C.), confirming their liquid nature at room temperature. Viscosity measurements also characterize these resins. For example, at 25° C., viscosities might be around 1397 cP for ZET, 426 cP for ZES, and 2208 cP for ZUR.

### Photopolymerizable Resin Compositions and 3D Printing Process

The invention further provides photopolymerizable resin compositions for 3D printing, which comprise at least one of the zingerol derivative monomers or crosslinkers, for example ZET, ZES, or ZUR. These compositions are typically formulated by mixing a zingerol derivative monomer or crosslinker with a photoinitiator. A common photoinitiator used in embodiments is diphenyl(2,4,6-trimethylbenzoyl)-phosphine oxide (TPO), often at a concentration of about 0.5% to 2% w/w, for example, about 1% w/w, relative to the monomer. In some cases, such as for the ZUR resin which might be highly reactive, a photoinhibitor may also be included in the formulation. These resin compositions of the present invention are typically solvent-free, which is advantageous for 3D printing and biomedical applications.

The method of manufacturing a 3D object using these compositions involves providing such a photopolymerizable resin composition and then exposing said composition to light in a layer-by-layer manner to form the 3D object. A preferred 3D printing technique is Digital Light Processing (DLP), which utilizes a digital projector to selectively cure layers of the resin.

Prior to printing complex objects, printing parameters are often optimized by establishing working curves for each resin formulation. This involves printing a calibration object (e.g., a CAD design with features of varying thicknesses) with varying exposure energies and measuring the resulting cure depths C_{d}. The working curve, plotting C_{d} as a function of exposure energy (Eₘₐₓ), allows for the determination of key printing parameters like penetration depth (Dₚ) and critical energy (E_{c}) for the resin. For example, ZES resin might show a penetration depth of around 0.30 mm, while ZET and ZUR might have Dₚ values of about 0.34 mm and 0.41 mm, respectively. E_{c} values might be around 4.23 mJ/cm² for ZET, 10.46 mJ/cm² for ZES, and 12.1 mJ/cm² for ZUR. Based on these parameters, optimal print settings (e.g., burn-in intensity, layer exposure intensity, exposure time per layer) are determined for each resin to achieve high-resolution printing.

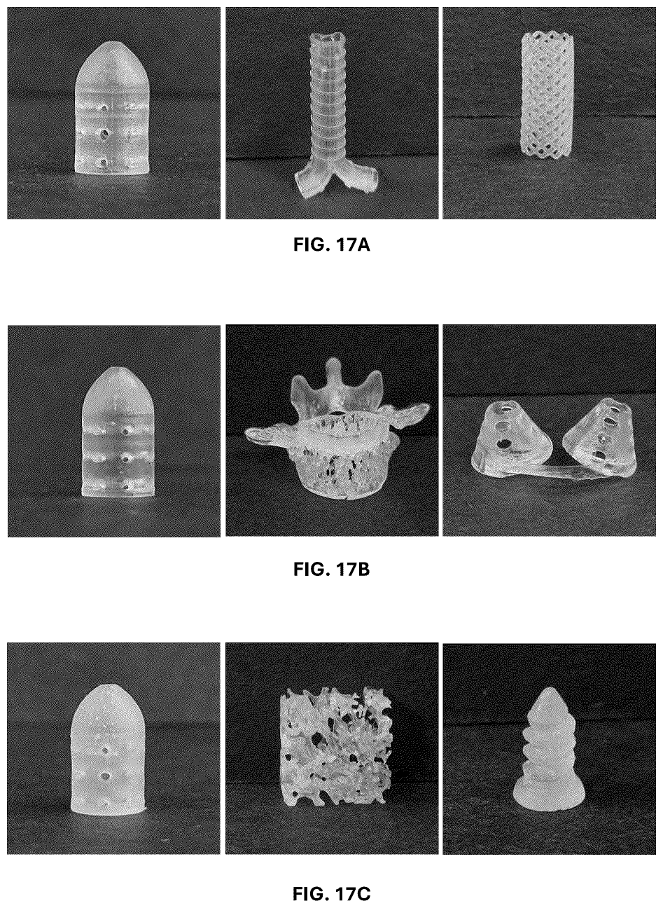

Using such optimized parameters, various complex three-dimensional objects can be fabricated from the ZET, ZES, and ZUR resins, as illustrated by the photographs in FIG. 17A (ZET objects), FIG. 17B (ZES objects), and FIG. 17C (ZUR objects). These can include models of medical devices, cylinders, cubes, catheters, stents, and other intricate shapes relevant to biomedical applications. After printing, the objects are typically removed from the printing platform, rinsed (e.g., with isopropanol and acetone) to remove unreacted resin, and then post-cured, for instance, using a UV flash machine (e.g., for about 30 minutes), to ensure complete polymerization and optimal material properties.

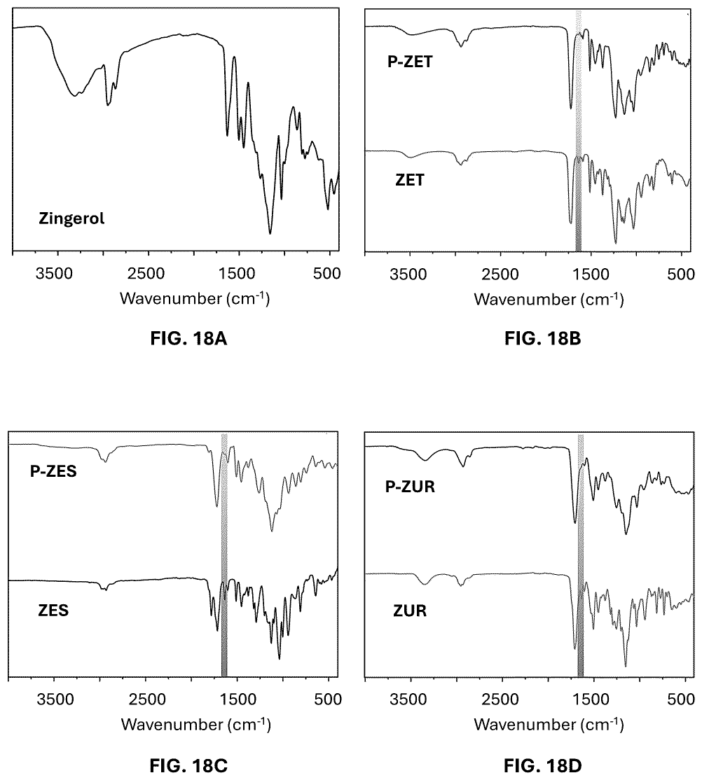

The quality of the 3D-printed objects can be further assessed. Reference is now made to FIGS. 18A-18D showing comparative FTIR spectra of zingerol (FIG. 18A) and of ZET (FIG. 18B), ZES (FIG. 18C) and ZUR (FIG. 18D) before and after their photopolymerization. The FTIR spectra of the printed polymers confirms successful photopolymerization of P-ZET, P-ZES and P-ZUR by showing the disappearance or significant reduction of the characteristic (meth)acrylate C═C double bond peaks (around 1640 cm⁻¹).

The quality of the 3D-printed objects was further characterized. For instance, evaluation of the printed P-ZET, P-ZES, and P-ZUR materials demonstrated good surface smoothness and print fidelity, consistent with the high-resolution capabilities of the employed 3D printing process. Microscopic analysis was also performed to confirm the achieved z-resolution in the layer-by-layer fabrication. In exemplary embodiments, layer heights for P-ZET and P-ZES prints were approximately 0.05 mm, while P-ZUR prints, which may possess different curing dynamics, were successfully fabricated with well-defined structures using layer heights of, for example, approximately 0.1 mm.

These observations indicate good control over the manufacturing process for these novel zingerol-based resins, despite variations in their specific compositions and resulting characteristics such as viscosity or potential for intermolecular interactions like hydrogen bonding in the case of P-ZUR.

### Characterization and Properties of 3D-Printed Objects (P-ZET, P-ZES, P-ZUR)

The 3D objects fabricated by the photopolymerization of the zingerol derivative resin compositions exhibit a compelling array of physicochemical, mechanical, and biological properties. These properties are extensively characterized to demonstrate the utility and advantages of these novel materials for biomedical applications. The polymerized materials are hereinafter referred to as P-ZET, P-ZES, and P-ZUR, corresponding to the polymers derived from the ZET, ZES, and ZUR zingerol derivative monomers, respectively.

In various embodiments, the successful photopolymerization and formation of a crosslinked polymer network are confirmed by spectroscopic techniques. As illustrated in FIGS. 18A-18D, the FTIR spectra of the 3D-printed P-ZET, P-ZES, and P-ZUR materials demonstrate the significant reduction or complete disappearance of the characteristic absorption peaks corresponding to the (meth)acrylate C═C double bonds (typically observed around 1630-1650 cm⁻¹ in the unpolymerized resins). This spectral change provides clear evidence of the consumption of these reactive groups during the light-induced polymerization process, leading to the formation of the polymeric network.

The thermal properties of these 3D-printed polymers are critical for their stability and behaviour under physiological or processing conditions. TGA and DSC are employed for this purpose with representative DSC thermograms and TGA/DTG curves for P-ZET, P-ZES, and P-ZUR shown in FIGS. 16E and 16F. In general, these photo-polymerized materials exhibit good thermal stability, often being stable up to temperatures around 250° C. before the onset of significant thermal degradation.

The TGA curves typically show a multi-stage degradation profile, which can be attributed to the decomposition of different structural components of the crosslinked zingerol-based network. For instance, main decomposition stages might be observed around 350° C. and further around 400° C., leading to substantial weight loss. In some embodiments, the P-ZET material, characterized by its ether linkages, may exhibit somewhat enhanced thermal stability at very high temperatures when compared to P-ZES and P-ZUR.

Due to their highly crosslinked thermoset nature, these 3D-printed polymers do not typically exhibit a distinct melting point (Tₘ) in their DSC thermograms. However, they possess well-defined glass transition temperatures (T_{g}), which are indicative of the transition from a glassy to a rubbery state and significantly influence their mechanical behavior and shape memory properties.

In specific embodiments, the T_{g} for P-ZET is found to be relatively low, for example, around 3.07° C. In contrast, P-ZES and P-ZUR exhibit much higher T_{g} values, for instance, around 116.87° C. for P-ZES and around 144.25° C. for P-ZUR. The significantly higher T_{g} of P-ZUR is attributed to its more rigid structure, likely due to the presence of urethane groups which can participate in extensive hydrogen bonding, in addition to the chemical crosslinks. Conversely, the lower T_{g} of P-ZET is consistent with the presence of more flexible ether bonds within its structural units, contributing to its elastomeric nature at physiological temperatures.

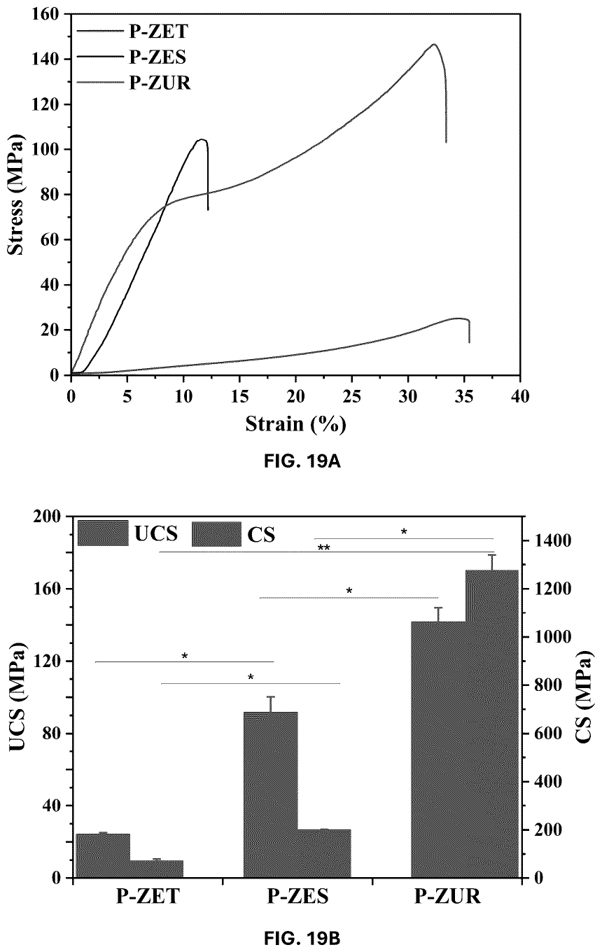

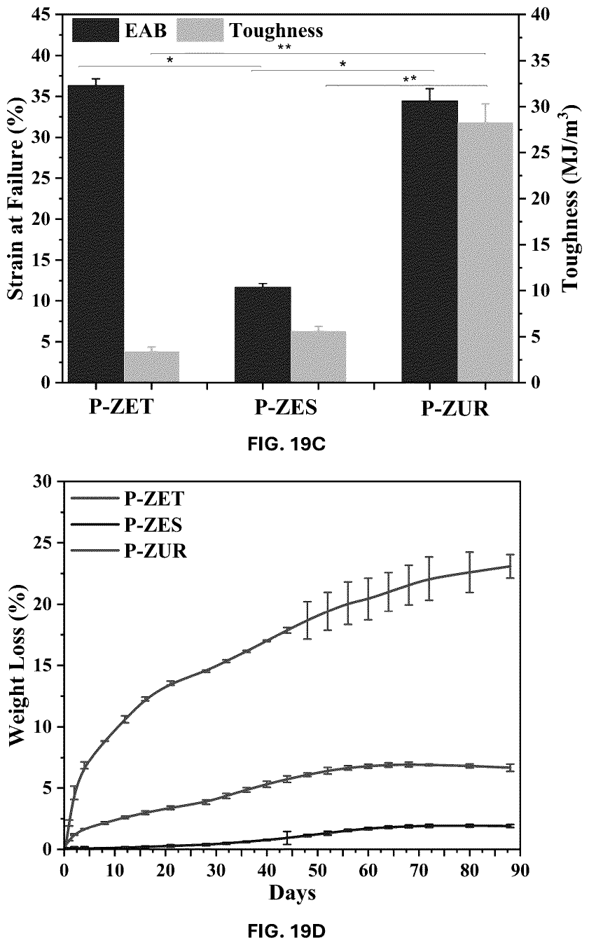

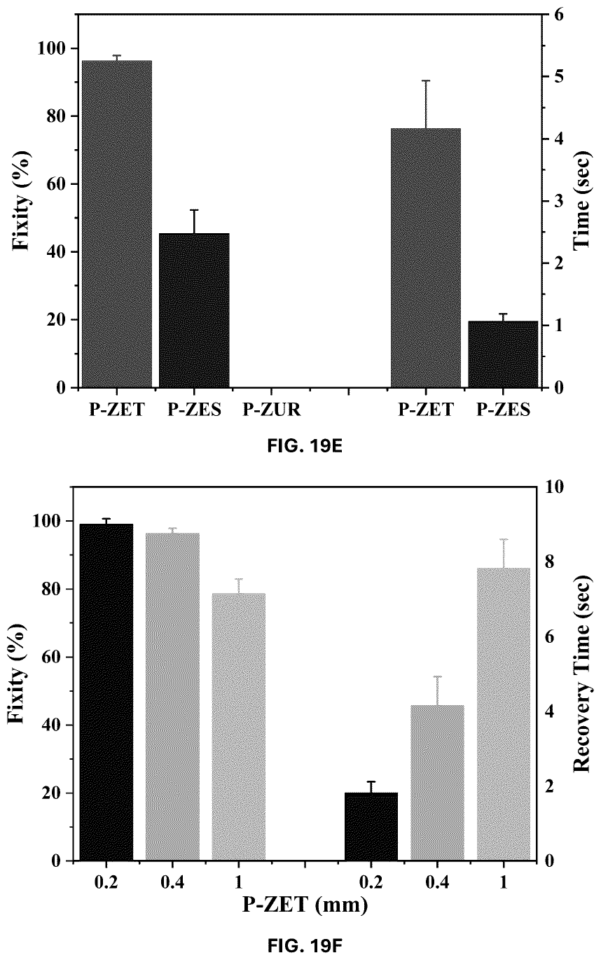

The mechanical properties of 3D-printed P-ZET, P-ZES, and P-ZUR objects are thoroughly investigated, as these are paramount for their intended use as load-bearing implants or scaffolds. These properties are typically assessed using compressive testing. Reference is now made to FIG. 19A showing the representative compressive stress-strain curves for P-ZET, P-ZES, and P-ZUR. The choice of the specific zingerol derivative (ZET, ZES, or ZUR) allows for significant tuning of these mechanical characteristics, enabling the materials to potentially mimic the properties of various human bones and connective tissues.

In certain illustrative embodiments, P-ZUR demonstrates the highest ultimate compressive strength (UCS), which may be in the order of approximately 141.43±8.11 MPa. P-ZES can exhibit an intermediate UCS, for example, around 91.43±8.72 MPa, while P-ZET, being more flexible, typically shows a lower UCS, for instance, about 24.04±0.99 MPa. The bar graph in FIG. 19B comparatively shows this trend. The superior strength of P-ZUR is attributed to the rigid urethane linkages and their capacity for strong intermolecular hydrogen bonding within the crosslinked network. The compressive stiffness (CS), or compressive modulus, also follows a similar trend, with P-ZUR exhibiting the highest stiffness (e.g., about 1276.54±62.57 MPa), followed by P-ZES (e.g., about 201.07±0.62 MPa), and then P-ZET (e.g., about 72.58±6.54 MPa), as also depicted in FIG. 19B.

In terms of maximum compressive strain at failure, shown in FIG. 19C, the polymer P-ZET often exhibits the highest strain capacity (e.g., about 36.34±0.79%), reflecting its more elastomeric nature. P-ZUR can also withstand significant strain before failure (e.g., about 34.44±1.47%), while P-ZES may exhibit a lower strain at failure (e.g., about 11.64±0.4%). The toughness of these materials, representing their ability to absorb energy under compression, is also shown in FIG. 19C. P-ZUR generally demonstrates the highest toughness (e.g., about 28.25±2.05 MJ/m³), while P-ZES and P-ZET exhibit toughness values of about 5.58±0.54 MJ/m³ and 3.37±0.51 MJ/m³, respectively.

The range of these mechanical properties is significant, as different values align well with those of various human tissues; for instance, the compressive strength and stiffness of P-ZUR are comparable to those of femur or cortical bone, P-ZES may mimic vertebrae, and P-ZET can be similar to trabecular (cancellous) bone or even softer tissues like nasal cartilage or knee ligaments, depending on the specific loading conditions and comparisons.

The biodegradability of these 3D-printed polymers is another key attribute for their application as temporary implants that can be gradually replaced by native tissue without the need for a second surgical intervention for removal. As illustrated by the weight loss profiles in FIG. 19D, in-vitro hydrolytic degradation studies, typically conducted in phosphate-buffered saline (DPBS, pH 7.4) at 37° C. over extended periods, demonstrate that P-ZET, P-ZES, and P-ZUR undergo gradual degradation. The rate and extent of degradation are dependent on the specific chemical backbone of the polymerized zingerol derivative. For instance, in some embodiments, P-ZET may exhibit a relatively more pronounced initial degradation rate compared to P-ZES and P-ZUR. P-ZET might show around 15% weight loss within the first 30 days, progressing to about 25% by 60 days. This behaviour can be correlated with its higher water uptake capacity (e.g., P-ZET absorbing approximately 9% water over 5 days, compared to about 1.96% for P-ZES and 4.17% for P-ZUR).

This higher water absorption in P-ZET may be attributed to the presence of free hydroxyl groups that are part of its structure (originating from the ring-opening of the glycidyl moiety of GMA during ZET synthesis) which are absent in P-ZES and P-ZUR structures derived from MA and IEMA respectively. P-ZUR, in some studies, shows a moderate degradation (e.g., about 4% in 30 days), while P-ZES might exhibit the slowest degradation profile among the three, particularly in the initial phases. The ability to tailor the degradation rate by choosing the appropriate zingerol derivative is advantageous for matching the resorption of the implant with the rate of tissue regeneration for specific clinical needs.

A particularly remarkable property exhibited by certain embodiments of these 3D-printed materials, especially P-ZET, is their excellent shape memory behaviour. This property allows a deformed object to recover its original, permanent shape upon application of an external stimulus, such as heat, which is highly valuable for developing minimally invasive surgical implants that can be delivered in a temporary, compact form. The shape memory performance is typically quantified by parameters such as shape fixity ratio (the ability to retain the temporary shape) and shape recovery ratio (the extent of return to the original shape).

As demonstrated in FIG. 19E, P-ZET can exhibit an excellent shape fixity ratio, often greater than 90%, and a shape recovery ratio of substantially 100%. The recovery can be quite rapid; for example, upon immersion in a 37° C. water bath (a physiologically relevant temperature), P-ZET samples may recover their shape in as little as approximately 2 to 4 seconds, with the exact time being dependent on the thickness of the printed part. P-ZES may also demonstrate good shape recovery (e.g., 100% recovery in about 2 seconds), whereas P-ZUR, with its more rigid structure and higher T_{g}, typically lacks significant shape memory properties under similar testing conditions. The superior shape memory effect observed in P-ZET is primarily attributed to its relatively low glass transition temperature (T_{g}≈3.07° C.), which is below body temperature, allowing for chain mobility and shape recovery, combined with the presence of flexible ether bonds in its backbone and a suitable crosslinked network structure that stores the permanent shape.

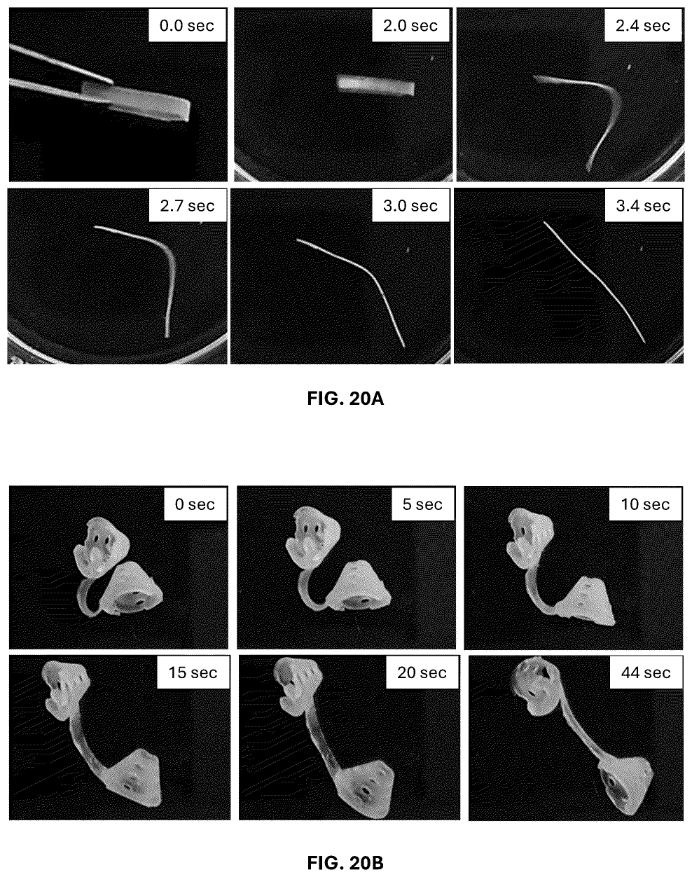

The influence of print thickness on the shape memory response of P-ZET is further illustrated in FIG. 19F, where it is shown that an increase in thickness generally leads to an increase in the recovery time (e.g., from about 1.5 seconds for a 0.2 mm thick P-ZET strip to about 8 seconds for a 1 mm thick strip), potentially due to increased mass and thermal transfer limitations, or denser hydrogen bonding networks in thicker prints. The visual demonstration of this shape memory capability is compelling, as shown in the series of digital photographic images in FIG. 20A, which document the rapid recovery of a deformed P-ZET strip to its original flat shape when placed in warm water. The practical application of this property is exemplified in FIG. 20B, which shows the shape recovery of a more complex 3D-printed object, such as a model of a nasal vestibular implant. This model implant may be fabricated using a combination of materials, for instance, with an exterior made from flexible, shape-memory P-ZET and an interior made from cytocompatible P-ZES, demonstrating the potential for creating sophisticated, functional medical devices.

Biological properties of the 3D-printed P-ZET, P-ZES, and P-ZUR materials are extensively investigated to ensure their suitability for biomedical applications, with key findings related to cytocompatibility, hemocompatibility, antioxidant activity, and antibacterial/anti-biofilm efficacy. Regarding cytocompatibility, these materials are evaluated with various relevant human and animal cell lines, including NIH-3T3 murine fibroblasts, HaCaT human keratinocytes (skin cells), and BEAS-2B human bronchial epithelial cells (lung cells). Studies involve assessing cell response both in direct contact with the surface of the printed materials and with conditioned media (extracts) derived from these materials.

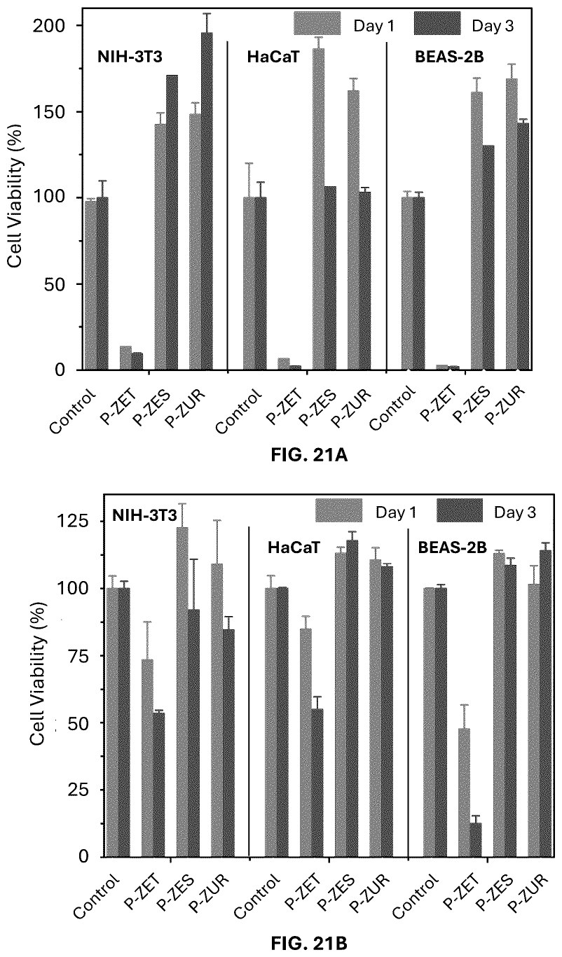

As shown by quantitative cell viability assays (e.g. Alamar Blue assay) shown in FIG. 21A (direct contact) and FIG. 21B (indirect contact), P-ZES and P-ZUR generally demonstrate excellent cytocompatibility. Cells cultured in the presence of P-ZES and P-ZUR often exhibit enhanced proliferation compared to control cultures, with cell viability frequently exceeding 100-140% relative to controls after 1 day of exposure.

This high level of biocompatibility, indicated by the quantitative cell viability assays (FIGS. 21A and 21B), was further corroborated by qualitative assessments using live/dead cell staining and fluorescence microscopy to examine cell morphology. These microscopic evaluations, conducted for cells in both direct contact with P-ZES and P-ZUR materials and for cells exposed to conditioned media from P-ZET, P-ZES, and P-ZUR (across NIH-3T3, HaCaT and BEAS-2B cell lines at, 1 and 3 day time points, for instance), consistently revealed a high preponderance of viable cells, indicated by strong Calcein-AM (green) fluorescence, and minimal numbers of dead cells, indicated by negligible ethidium homodimer-1 (red) fluorescence, in cultures exposed to P-ZES and P-ZUR. Similarly, cells exposed to conditioned media from P-ZET also generally maintained high viability, particularly in indirect contact assays with NIH-3T3 and HaCaT cells.

These observations were comparable to control cell populations under normal culture conditions, affirming the excellent cytocompatibility of these zingerol-based materials, especially P-ZES and P-ZUR. These images typically show a high population of viable cells (e.g., stained green with Calcein-AM) and very few dead cells (e.g., stained red with ethidium homodimer-1) when cultured with P-ZES and P-ZUR. Cell morphology analysis from these studies, often involving staining for F-actin filaments and nuclei (DAPI), further reveals that cells maintain a healthy, well-spread phenotype with normal cytoskeletal organization and intercellular connections, indicating that these materials do not adversely affect cell structure or function.

In some embodiments, P-ZET may exhibit a degree of cytotoxicity when cells are in direct contact with its surface, potentially related to its initial degradation products or surface chemistry. However, P-ZET usually shows good cytocompatibility in indirect contact tests (i.e., with conditioned media), particularly with NIH-3T3 and HaCaT cells, although it may show lower compatibility with BEAS-2B lung cells in some conditions. Overall, P-ZES and P-ZUR stand out for their superior cytocompatibility across multiple cell lines and conditions.

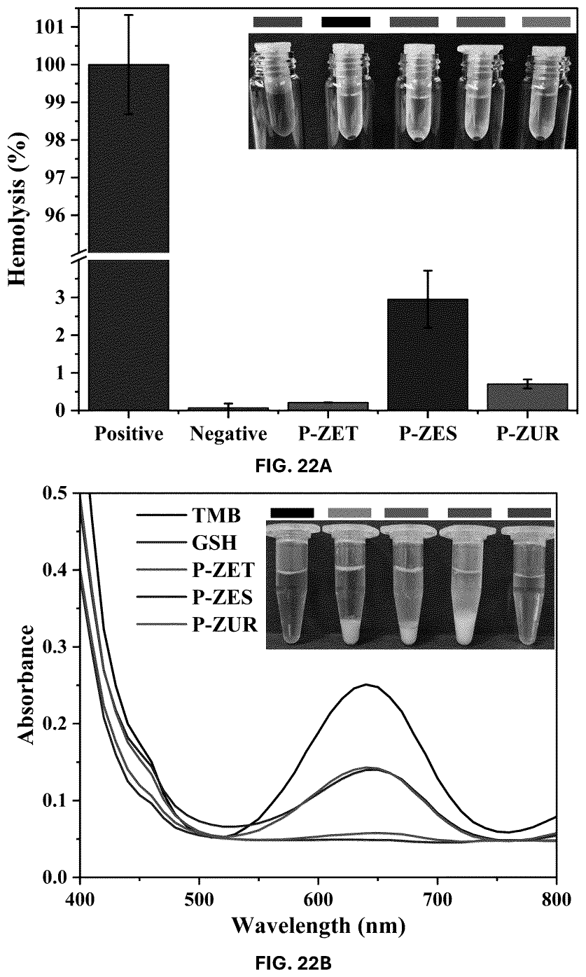

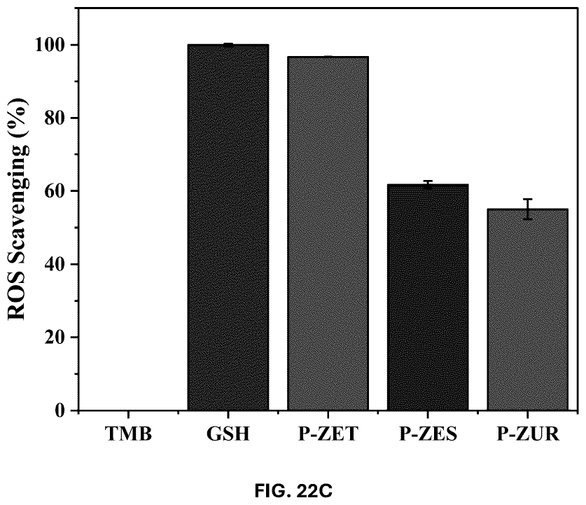

In terms of hemocompatibility, the P-ZET, P-ZES, and P-ZUR prints are evaluated for their interaction with human blood, specifically their potential to cause haemolysis (rupture of red blood cells). As demonstrated by the results presented in FIG. 22A, all three types of printed materials typically exhibit very low haemolysis rates when incubated with human red blood cells. For instance, haemolysis percentages are generally well below the clinically acceptable limit of 5%, often less than 3% (e.g., P-ZUR showing around 2.95% haemolysis, while P-ZES and P-ZET may show even lower values, often less than 1%). This indicates that these materials are substantially hemocompatible and pose a low risk of adverse haematological reactions if used in applications involving direct or indirect blood contact.

The 3D-printed zingerol-based materials of the invention also possess significant antioxidant efficacy, which is a valuable property for biomaterials intended for implantation, as oxidative stress is often implicated in inflammatory responses to foreign materials and in various pathological conditions. This antioxidant potential is likely derived from the zingerol moiety.

The ROS scavenging capability is demonstrated through assays such as the 3,3′,5,5′-tetramethylbenzidine (TMB) assay. As shown by the UV-Vis spectra and corresponding photographic images of the solutions in FIG. 22B, the printed materials (P-ZET, P-ZES, P-ZUR) can effectively scavenge hydroxyl radicals (generated, for example, from H₂O₂ in the presence of Fe³⁺), thereby inhibiting the oxidation of TMB and reducing the formation of the characteristic blue-coloured oxidation product. Quantitative analysis of these results, presented as the ROS scavenging percentages in FIG. 22C, shows that P-ZET, in particular, can exhibit excellent ROS scavenging activity, for instance, around 96.72%, which is comparable to that of standard antioxidants like glutathione (GSH). P-ZES and P-ZUR also demonstrate good scavenging activity, for example, around 61.71% and 55%, respectively.

Furthermore, the ability of these materials to counteract oxidative stress at a cellular level is demonstrated by intracellular ROS scavenging assays, such as the dichlorodihydrofluorescein diacetate (DCFDA) assay conducted with cells like NIH-3T3 murine fibroblasts. Furthermore, the ability of these materials to counteract oxidative stress at a cellular level was demonstrated by intracellular ROS scavenging assays, such as the dichlorodihydrofluorescein diacetate (DCFDA) assay conducted with NIH-3T3 murine fibroblast cells.

Microscopic evaluation in these assays revealed that pretreatment of cells with the printed materials, particularly P-ZET, led to a significant reduction in the fluorescence intensity indicative of intracellular ROS levels when cells were subsequently challenged with an oxidative agent like hydrogen peroxide (H₂O₂). This observation supports the capacity of these materials, especially P-ZET, to protect cells from oxidative damage by scavenging intracellular ROS induced by an oxidative agent like hydrogen peroxide (H₂O₂), indicating that these materials can protect cells from oxidative damage.

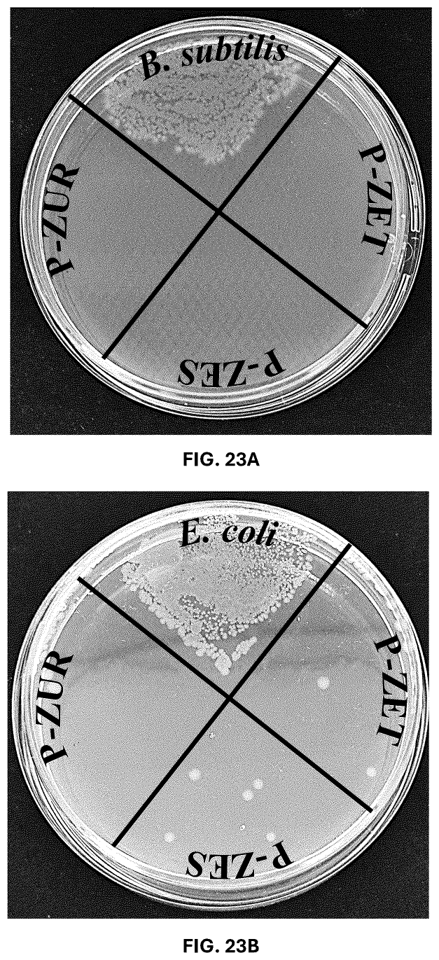

A particularly important and advantageous characteristic of these 3D-printed zingerol-based materials is their potent antibacterial and anti-biofilm efficacy. The prevention of bacterial colonization and biofilm formation on implant surfaces is a major challenge in medicine. The P-ZET, P-ZES, and P-ZUR prints demonstrate significant activity against both Gram-positive bacteria (e.g., Bacillus subtilis) and Gram-negative bacteria (e.g., Escherichia coli). This antibacterial effect is evidenced by colony formation assays, as shown by the photographic images of agar plates in FIG. 23A (for B. subtilis) and FIG. 23B (for E. coli), where direct contact with the printed material surfaces leads to a marked reduction or complete inhibition of bacterial colony growth compared to control surfaces.

The bactericidal nature of these materials, complementing the colony formation assay results (FIGS. 23A and 23B), was further confirmed by bacterial live/dead staining assays using fluorescence microscopy. These microscopic analyses for both B. subtilis and E. coli exposed to P-ZET, P-ZES, and P-ZUR consistently showed a high proportion of dead bacteria (e.g., indicated by red or yellow fluorescence) and a significant reduction in live bacteria (e.g., indicated by green fluorescence) compared to control bacterial populations, thereby visually verifying the potent antibacterial effect of the printed materials.

These images typically show a high proportion of dead bacteria (e.g., stained red/yellow) on or near the surfaces of the printed materials, in contrast to predominantly live bacteria (e.g., stained green) in control samples. SEM analysis of bacteria that have interacted with the P-ZET, P-ZES, and P-ZUR surfaces often reveals significant damage to the bacterial cell membranes, including evidence of membrane disruption, blebbing, or lysis. This suggests that the antibacterial mechanism involves direct interaction with and compromise of the bacterial cell envelope, a property likely contributed by the zingerol component, which is known to have membrane-active properties.

Beyond just killing planktonic bacteria, these materials are also highly effective in preventing the formation of bacterial biofilms, which are notoriously resistant to antibiotics and host defences. Beyond their effects on planktonic bacteria, these materials are also highly effective in preventing the formation of bacterial biofilms. This was demonstrated through experiments where B. subtilis and E. coli were cultured on the surfaces of P-ZET, P-ZES, and P-ZUR under conditions conducive to biofilm development.

Subsequent analysis, for example by SEM, revealed a significant inhibition or complete prevention of biofilm formation on the surfaces of the inventive materials. In contrast, control surfaces (such as Keysplint, a commercial dental implant material) typically showed extensive biofilm coverage under the same conditions, highlighting the potent anti-biofilm efficacy of the zingerol-based printed polymers. In contrast, control surfaces (such as Keysplint, a commercial dental implant material) may show extensive biofilm coverage under the same conditions. This potent anti-biofilm efficacy is a critical advantage for any material intended for use as a medical implant, as it can drastically reduce the risk of implant-associated infections and their severe consequences.

### Uses of 3D-Printable Zingerol-Derivative Compositions and Objects

The unique and advantageous combination of properties exhibited by the photopolymerizable zingerol derivative compositions and the 3D-printed objects derived therefrom makes them exceptionally promising for a wide spectrum of advanced applications, particularly within the biomedical field. The ability to fabricate intricate, patient-specific devices via 3D printing, coupled with the materials' inherent biocompatibility, tuneable mechanical characteristics, biodegradability, shape memory capabilities, and potent biological functionalities (such as antibacterial, anti-biofilm, and antioxidant activities), opens up numerous therapeutic and diagnostic possibilities.

In various embodiments of the invention, the 3D-printed objects are contemplated for use as medical implants or as scaffolds for tissue engineering. The high-resolution 3D printing capability, demonstrated by the fabrication of complex designs from ZET, ZES, and ZUR resins as shown above allows for the creation of customized, patient-specific implants. This is particularly relevant for orthopaedic applications (e.g., bone screws, plates, or porous bone void fillers), craniofacial reconstruction, and dental implants. For dental applications, the pronounced anti-biofilm efficacy of these materials is a significant advantage in preventing peri-implantitis and other infection-related complications. The tuneable mechanical properties, ranging from the flexible nature of P-ZET to the high compressive strength of P-ZUR (shown above), enable the matching of implant characteristics to the specific demands of the host tissue, for instance, P-ZUR for mimicking cortical bone or P-ZET for cartilage-like applications.

As scaffolds for tissue engineering, these 3D-printable zingerol-based materials offer several benefits. Their biocompatibility, as demonstrated by extensive cytocompatibility studies across multiple cell lines, ensures that they provide a favorable environment for cell attachment, proliferation, and differentiation. The 3D printing process allows for the precise design and fabrication of scaffolds with controlled porosity, interconnectivity, and micro-architecture, which are crucial for facilitating cell infiltration, nutrient transport, vascularization, and ultimately, new tissue formation.

For bone tissue engineering, in particular, materials like P-ZES and P-ZUR with bone-mimicking mechanical strength, coupled with the inherent antibacterial, anti-biofilm, and antioxidant properties of the zingerol derivatives, can contribute to creating a pro-regenerative microenvironment that supports osteogenesis while minimizing risks of infection and inflammation. The biodegradability of these scaffolds ensures that they can provide temporary support and then gradually resorb as the native tissue regenerates, obviating the need for subsequent removal surgeries.

In further embodiments, the 3D-printed objects of the invention are suitable for use as drug delivery systems. The polymer matrix can be designed to encapsulate therapeutic agents (e.g., antibiotics, anti-inflammatory drugs, growth factors) and release them in a controlled and sustained manner at the target site. This localized drug delivery can enhance therapeutic efficacy while minimizing systemic side effects. The inherent biological activities of the zingerol-based matrix itself (e.g., antibacterial, antioxidant) can act synergistically with the loaded drugs.

The remarkable shape memory properties exhibited by some embodiments, particularly those derived from P-ZET, enable the development of innovative devices for minimally invasive surgery (MIS). Implants or devices can be fabricated in a specific functional shape, then temporarily deformed into a compact, compressed form for easy insertion through small incisions or catheters. Once in situ, exposure to body temperature (around 37° C.) can trigger the material to recover its original, pre-programmed functional shape. This is exemplified by the potential application in flexible implants such as nasal vestibular stents, where a combination of shape-memory P-ZET and cytocompatible P-ZES can be utilized to create devices that are easily deployed and adapt to anatomical contours. This capability can significantly reduce patient trauma, shorten recovery times, and allow for surgical interventions in otherwise difficult-to-access areas.

Moreover, the inherent biological functionalities of these zingerol-based materials allow for their targeted use in applications specifically requiring antibacterial and anti-biofilm surfaces. This includes not only implants but also coatings for existing medical devices, components of catheters, wound dressings, or any surface where preventing microbial colonization is critical. The demonstrated efficacy against both Gram-positive and Gram-negative bacteria, and importantly, against biofilm formation, makes these materials highly valuable in combating device-associated infections.

Similarly, applications where local antioxidant and potentially anti-inflammatory effects are desired can benefit from these materials. The ability to scavenge reactive oxygen species (ROS), as demonstrated in the present invention, can help mitigate oxidative stress at the implant site or in a wound bed, thereby modulating the host's inflammatory response and promoting a more favorable healing environment. This can be particularly useful for implants in tissues prone to inflammation or for advanced wound care products.

In summary, the 3D-printable zingerol derivative compositions and the objects manufactured therefrom, according to various aspects of the invention, represent a significant advancement in the field of biomedical materials. Their unique combination of 3D printability into complex and patient-specific forms, tunable physicochemical and mechanical properties, biodegradability, inherent biological functionalities (antibacterial, anti-biofilm, antioxidant), excellent biocompatibility, and shape memory characteristics positions them as highly versatile and promising candidates for a wide range of therapeutic devices and regenerative medicine strategies. The method of manufacturing these 3D objects, involving providing the photopolymerizable resin composition and exposing it to light in a layer-by-layer manner, enables the practical realization of these advanced biomedical applications.

### CONCLUSIONS

The present invention provides a significant advancement in the field of biomedical materials via the innovative utilization of renewable and biocompatible compound derived from ginger) to create two distinct yet complementary classes of advanced functional polymeric materials. These materials, encompassing both biodegradable polyesters and 3D-printable photopolymerizable compositions, offer a unique combination of tuneable physicochemical properties, inherent t biological functionalities and environmentally considerate synthesis methodologies, addressing critical unmet needs in regenerative medicine, medical implant technology, and advanced drug delivery.

In one major aspect, the invention details the first-time synthesis of novel biodegradable polyesters, exemplified by the ZCSX series (which utilizes zingerol), through a unique solvent-free and catalyst-free melt polycondensation process applicable to the broader class of claimed plant-derived phenolic diols. This environmentally friendly approach yields polyesters whose mechanical properties, degradation rates, and shape memory characteristics can be precisely tuned by adjusting monomer ratios.

These ZCSX polyesters have demonstrated excellent cytocompatibility, hemocompatibility, inherent antibacterial activity against both Gram-positive and Gram-negative bacteria, and a promising potential to promote wound healing. Their mechanical resemblance to various human soft tissues, coupled with their robust shape memory behaviour, makes them highly suitable for applications such as soft tissue engineering scaffolds and devices for minimally invasive surgery.

The novelty here lies not only in the specific polyester compositions derived from zingerol but also in the advantageous green synthesis route that enhances their biomedical applicability by minimizing potential toxic residues.

In a second major aspect, the invention introduces, also for the first time, unique ginger-based (zingerol-derived) photopolymerizable resin compositions, specifically Zingerol-glycidyl methacrylate (ZET), Zingerol-methylacrylate (ZES), and Zingerol-urethane (ZUR). These novel zingerol derivatives are ingeniously designed to be photopolymerizable, enabling their formulation into solvent-free resins for high-resolution 3D printing of complex, patient-specific medical devices and scaffolds.

The resulting 3D-printed objects (P-ZET, P-ZES, P-ZUR) exhibit a remarkable confluence of properties: their mechanical characteristics can be tailored to mimic various human tissues ranging from flexible cartilage (P-ZET) to strong bone (P-ZUR); they are biodegradable; they demonstrate exceptional cytocompatibility with multiple human cell lines and are highly hemocompatible. Most notably, these 3D-printed materials retain and amplify the beneficial biological activities derived from the zingerol core, exhibiting potent antioxidant effects by scavenging reactive oxygen species, significant antibacterial activity, and, critically, outstanding anti-biofilm efficacy against common pathogenic bacteria.

Furthermore, specific compositions like P-ZET display excellent thermoresponsive shape memory properties, ideal for deployable medical devices. The novelty of this aspect resides in the creation of these specific photopolymerizable zingerol derivatives, their formulation into 3D-printable resins, and the unprecedented combination of 3D printability, tunable mechanics, biodegradability, and potent multifunctionality (antibacterial, anti-biofilm, antioxidant, shape memory) in the final printed objects.

The unity of invention between these two aspects—the polyesters derived from the claimed class of plant-derived phenolic diols (with ZCSX from zingerol as a key example) and the 3D-printable P-ZET/P-ZES/P-ZUR materials derived from zingerol—is firmly established through the central, inventive use of these specific types of plant-derived phenolic diols, particularly zingerol, as the foundational building block for creating advanced functional polymers via distinct synthetic strategies tailored for biomedical applications.

The present invention ingeniously leverages zingerol, a single, naturally derived, biocompatible, and inherently functional diol, to create two distinct families of polymeric materials through two different innovative synthetic strategies (environmentally friendly polycondensation for one, and novel derivatization for photopolymerization for the other). Both strategies transform zingerol into advanced polymers tailored for biomedical challenges.

While the ZCSX polyesters explore the direct incorporation of zingerol into polyester backbones to achieve one set of desired functionalities (e.g., specific elasticity, shape memory via polyester network design), the ZET/ZES/ZUR derivatives re-engineer the zingerol molecule itself into novel photopolymerizable monomers, enabling a different set of applications focused on 3D printing and yielding materials with properties like superior anti-biofilm efficacy. Thus, the overarching inventive concept is the versatile and novel exploitation of zingerol to develop a platform of multifunctional polymeric biomaterials, addressing diverse needs within the biomedical field through tailored chemical approaches.

The novelty and inventiveness of the present invention are further underscored when considering the solutions it offers to long-felt needs. For example, consider a patient requiring a custom orthopaedic implant to replace a section of bone damaged by trauma or disease. Current metallic or inert polymer implants often come in standard sizes, may require significant surgical adaptation, carry a persistent risk of bacterial infection leading to biofilm formation and implant failure, and can elicit chronic inflammatory responses.

The present invention offers a transformative alternative: Using the patient's specific anatomical data, a precisely fitting implant can be designed and then 3D-printed using, for instance, the P-ZUR resin composition where high mechanical strength is paramount for applications like robust bone replacement; the P-ZES resin composition, which provides a compelling combination of moderate stiffness, excellent cytocompatibility, sustained anti-biofilm activity, and potentially longer-term structural support due to its favorable degradation profile; or a composite incorporating P-ZET where significant flexibility or specific shape-memory responses are critical.

This 3D-printed zingerol-based implant would not only be perfectly customized but would also inherently combat bacterial colonization due to its anti-biofilm properties, reduce oxidative stress and inflammation at the implant site due to its antioxidant nature, and eventually biodegrade safely as new bone tissue regenerates, potentially negating the need for a second removal surgery. This single implant thus addresses multiple critical challenges-customization, infection, inflammation, and implant permanence-all stemming from the inventive use and modification of zingerol.

In conclusion, the present invention provides a significant contribution to the art by introducing two novel platforms of zingerol-based polymeric biomaterials. These materials, produced via distinct and innovative synthetic routes, offer a rich palette of tuneable physical properties and potent biological functionalities. They overcome critical limitations of existing materials and open new avenues for developing advanced, patient-specific solutions for tissue engineering, medical implants, drug delivery, and other biomedical applications, all while leveraging a safe, renewable, natural-product-derived core molecule. The disclosed polyesters and 3D-printable compositions represent a paradigm shift towards truly multifunctional, intelligent biomaterials.

1. A polyester polymer comprising at least one chain having Formula (I): 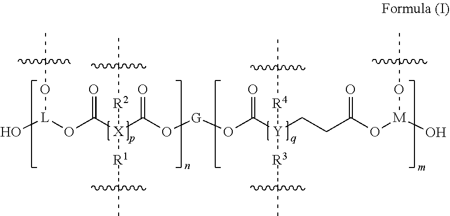
wherein —O-G-O— represents a plant-derived diol moiety G selected from the group consisting of gingerols, zingerol, and reduced derivatives of paradol, zingerone, and shogaols,
X is a methylene or methylidene group of a polycarboxylic acid, said methylene or methylidene group is optionally substituted with R¹ and/or R² group, each independently selected from the group consisting of hydroxyl, carboxyl, carboxylate, (C₁-C₁₀)-alkanol, (C₁-C₁₀)-alkyl-carboxyl, and (C₁-C₁₀)-alkyl-carboxylate,
Y is a methylene or methylidene group of a polycarboxylic acid, said methylene or methylidene group is optionally substituted with R³ and/or R⁴ group, each independently selected from the group consisting of hydroxyl, carboxyl, carboxylate, (C₁-C₁₀)-alkanol, (C₁-C₁₀)-alkyl-carboxyl, and (C₁-C₁₀)-alkyl-carboxylate,
L and M are the same or different polyol moieties linked via an ester bond to said groups X and Y, respectively, through one of their hydroxyl groups,
n and m are integers independently selected from 1 to 10,000, and
p and q are integers independently selected from 1 to 24;
wherein said at least one chain is optionally cross-linked via an ester bond to at least one other chain through hydroxyl groups of said polyol moieties L and/or M, and/or through groups R¹, R², R³ and/or R⁴ of groups X and Y, respectively, and
wherein ‘ [symbol] ’ denotes an optional covalent bond.

2. The polyester polymer of claim 1, wherein said plant-derived diol is zingerol.

3. The polyester polymer of claim 2, wherein said zingerol is derived from zingerone by reduction.

4. The polyester polymer of claim 1, wherein the polycarboxylic acid residues from which X and Y are derived are obtained from at least one polycarboxylic acid selected from the group consisting of sebacic acid and citric acid.

5. The polyester polymer of claim 1, wherein at least one of said polyol moieties L or M is derived from xylitol, and wherein said xylitol functions as a crosslinker.

6. The polyester polymer of claim 1, wherein said plant-derived diol moiety G is derived from zingerol, the polycarboxylic acid residues from which X and Y are derived are obtained from sebacic acid and citric acid, and at least one of said polyol moieties L or M is derived from xylitol.

7. A method for preparing a polyester polymer as defined in claim 1, said method comprising:
(i) reacting a plant-derived diol, from which moiety G is derived, of the formula: 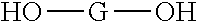
at least one polycarboxylic acid, from which residues X and Y are derived, of the formula: 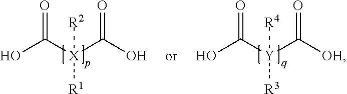
and
optionally at least one polyol, from which moieties L and M are derived, of the formula: 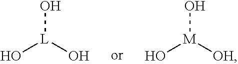
in a first step via a catalyst-free and solvent-free melt polycondensation to form a pre-polymer; and
(ii) curing said pre-polymer to form the biodegradable polyester polymer;
where X is a methylene or methylidene group of a polycarboxylic acid, said methylene or methylidene group is optionally substituted with R¹ and/or R² group, each independently selected from the group consisting of hydroxyl, carboxyl, carboxylate, (C₁-C₁₀)-alkanol, (C₁-C₁₀)-alkyl-carboxyl, and (C₁-C₁₀)-alkyl-carboxylate,
Y is a methylene or methylidene group of a polycarboxylic acid, said methylene or methylidene group is optionally substituted with R³ and/or R⁴ group, each independently selected from the group consisting of hydroxyl, carboxyl, carboxylate, (C₁-C₁₀)-alkanol, (C₁-C₁₀)-alkyl-carboxyl, and (C₁-C₁₀)-alkyl-carboxylate,
L and M are the same or different polyol moieties,
p and q are integers independently selected from 1 to 24; and
wherein ‘ [symbol] ’ denotes an optional covalent bond to an optional hydroxyl group of the polyol.

8. The method of claim 7, wherein the plant-derived diol is zingerol, the at least one polycarboxylic acid is selected from the group consisting of sebacic acid and citric acid, and the at least one polyol includes xylitol.

9. The polyester polymer of claim 1, for use in an application selected from the group consisting of a shape memory material, a material having antibacterial activity, regenerative medicine, wound healing, implant items, and tissue engineering.

10. A photopolymerizable zingerol derivative monomer or crosslinker having the structure as depicted in Formula (II): 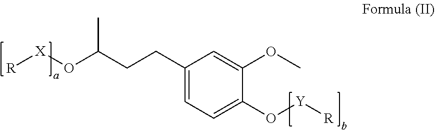
wherein the oxygen atom attached to X and the oxygen atom attached to Y are derived from the native secondary aliphatic hydroxyl group and the native phenolic hydroxyl group of zingerol, respectively;
a and b are independently 0 or 1, with the proviso that a+b is at least 1;
each R independently represents a photopolymerizable (meth)acrylate group of the formula 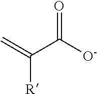
wherein R′ is H or methyl;
each X, when a is 1, independently represents a divalent linking group such that the moiety —O—X—R forms a functionalized zingerol aliphatic hydroxyl group wherein an ether, ester, or urethane linkage connects said oxygen to said R group via said linking group X; and
each Y, when b is 1, independently represents a divalent linking group such that the moiety —O—Y—R forms a functionalized zingerol phenolic hydroxyl group wherein an ether, ester, or urethane linkage connects said oxygen to said R group via said linking group Y.

11. The photopolymerizable zingerol derivative monomer or crosslinker of claim 10, wherein for at least one functionalized hydroxyl group:
(i) for an ester linkage, X or Y is a direct bond, resulting in a structure: 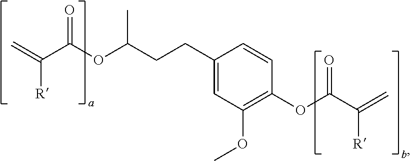
or
(ii) for an ether-type linkage derived from glycidyl (meth)acrylate, X or Y is —CH₂—CH(OH)—CH₂—O—; or
(iii) for a urethane linkage derived from an isocyanatoalkyl (meth)acrylate, X or Y is —C(═O)—NH—R″—O—, wherein R″ is a C₁-C₆ alkylene group.

12. The photopolymerizable zingerol derivative monomer or crosslinker of claim 11, selected from the group consisting of zingerol-glycidyl methacrylate (ZET), zingerol-methylacrylate (ZES), and zingerol-urethane (ZUR).

13. A photopolymerizable resin composition for 3D printing, comprising at least one zingerol derivative monomer or crosslinker as defined in claim 10, and optionally a photoinitiator.

14. A method for preparing a zingerol derivative monomer or crosslinker as defined in claim 10, said method comprising reacting zingerol with at least one reagent selected from the group consisting of glycidyl (meth)acrylate, (meth)acrylic anhydride, and an isocyanatoalkyl (meth)acrylate, to functionalize at least one hydroxyl group of said zingerol with a photopolymerizable (meth)acrylate group via an ether, ester, or urethane linkage.

15. A 3D-printed object comprising the polymerized product of the photopolymerizable resin composition of claim 13.

16. The 3D-printed object of claim 14, wherein the zingerol derivative monomer or crosslinker used to form the polymerized product is selected from the group consisting of zingerol-glycidyl methacrylate (ZET), zingerol-methylacrylate (ZES), and zingerol-urethane (ZUR).

17. The 3D-printed object of claim 15, wherein said object exhibits shape memory properties with greater than 90% fixity and substantially 100% recovery.

18. The 3D-printed object of claim 17, wherein the zingerol derivative monomer or crosslinker used to form the polymerized product is zingerol-glycidyl methacrylate (ZET).

19. The 3D-printed object of claim 15, wherein said object exhibits anti-biofilm efficacy against Gram-positive and Gram-negative bacteria.

20. The 3D-printed object of claim 15, wherein said object exhibits antioxidant properties by scavenging reactive oxygen species.

21. The 3D-printed object of claim 15, for use as a patient-specific medical implant or as a scaffold for bone tissue engineering.

22. A method of manufacturing a 3D object, comprising the steps of:
(a) providing the photopolymerizable resin composition of claim 13; and
(b) exposing said composition to light in a layer-by-layer manner to form the 3D object.
